(19) United States
(12) Patent Application Publication
(10) Pub. No.: US 2024/0272149 A1
(43) Pub. Date: Aug. 15, 2024

(54) DETECTION OF BIOACTIVE AGENTS IN A SURROUNDING MEDIUM

(71) Applicant: Philip Morris Products S.A., Neuchatel (CH)

(72) Inventor: Sandro STEINER, Neuchatel (CH)

(73) Assignee: Philip Morris Products S.A., Neuchatel (CH)

(21) Appl. No.: 18/681,989

(22) PCT Filed: Oct. 6, 2022
(86) PCT No.: PCT/EP2022/077874

§ 371 Date: Feb. 7, 2024

(30) Foreign Application Priority Data
Oct. 8, 2021 (EP) 21201778.4

(51) Int. Cl. G01N 33/543

(52) U.S. Cl. CPC G01N 33/54366

(57) ABSTRACT

A sensor chip and a sensing device containing the sensor chip is provided. The sensing device is configured to detect one or more bioactive agents in a surrounding medium. The sensor chip includes a reaction cell containing a plurality of receptor protein complexes, and a membrane separating the reaction cell from the surrounding medium. The membrane is permeable to the bioactive agents and the receptor protein complexes are configured to bind to the bioactive agents within the reaction cell to induce a state change of the receptor protein complexes which is detected.

The present disclosure generally relates to the detection of bioactive agents. In particular, the present disclosure relates to a sensor chip couplable to a sensing device and to a sensing device for detecting one or more bioactive agents in a surrounding medium. Further, the present disclosure relates to a sensing system comprising such sensor chip and sensing device. Moreover, the present disclosure relates to a use of such sensor chip, sensing device or sensing system and to a method of detecting one or more bioactive agents with such sensor chip, sensing device or sensing system.

Humans, animals, or living organisms in general continuously interact with their chemical environment. For example, living organisms are usually exposed to or in contact with a surrounding medium, such as surrounding air or water, and may receive, ingest, absorb or otherwise take up molecules, agents or compounds from the surrounding medium, thereby interacting with their chemical environment. Interactions related to particular molecules, agents or compounds from the surrounding medium may be desirable or even required for the organisms to survive. Examples of such interactions are oxygen that is inhaled by humans or animals and binds to haemoglobin in the red blood cells passing the pulmonary capillaries, or sugar molecules that are selectively transported across the intestinal epithelia. However, chemical interactions with the surrounding medium related to other molecules, agents or compounds may not be part of the normal, life sustaining processes and may have undesirable effects, potentially even causing harm to an organism. Such undesirable effects can be caused by molecules, agents or compounds received by the organism from the surrounding medium, which non-specifically damage the molecular building blocks of cells or the cells as a whole. For example, oxidizing compounds, such as ozone, may be taken-up by an organism and oxidize biomolecules of the organism, such as proteins, lipids or nucleic acids, and may decrease or even destroy their biochemical and/or structural functionality. Alternatively, chemical compounds, agents or molecules taken-up by the organism may activate, inhibit, or even eliminate a specific biochemical process of the organism, often without chemically changing biomolecules of the organism. Examples for such processes or interactions are the inhibition of the enzyme cyclooxygenase by ibuprofen or the activation of the nicotinic acetylcholine receptor by nicotine. Typically, such interactions are dependent on the structural match between the affected biomolecule and the effector molecule, agent or compound. Ibuprofen, for example, binds to the active site of cyclooxygenase, thereby preventing binding of arachidonic acid, which under normal circumstances is converted by the cyclooxygenase to signal molecules involved in the onset of inflammation and pain.

Living organisms have developed and evolved mechanisms for detecting harmful chemical compounds, agents or molecules they are exposed to and for triggering appropriate physiological responses. Chemical compounds, agents and molecules triggering a physiological response in a living organism may generally be referred to as bioactive agents herein. For humans and other living organisms, various receptor proteins have been identified, which serve for detecting bioactive agents and triggering a physiological response. Prominent examples of receptor proteins are the aryl hydrocarbon receptor (AHR), the pregnane X receptor (PXR) and the constitutive androstane receptor (CAR). These receptor proteins show a certain binding specificity towards bioactive agents, in particular so-called xenobiotics, which relate to bioactive agents, compounds or molecules that are foreign to biological systems. On the other hand, there are various receptor proteins that have not evolved to detect and defend against potentially harmful bioactive agents such as xenobiotics, but rather as part of pivotal regulatory systems. For instance, the endocrine or hormonal system controls various developmental and steady-state functions in animals or humans including growth, organogenesis, fertility, reproduction and sleep. These receptor proteins specifically bind and are activated by their cognate ligands, that is hormones. Bioactive agents sharing certain structural features with hormones may, however, bind and also activate the receptors. This process may be referred to as endocrine disruption and given the pivotal role of the endocrine system, may have harmful effects. For instance, exposure to xenoestrogens, which are bioactive agents or xenobiotic compounds activating the estrogen receptors (ER), have been attributed to the onset of breast, lung, kidney, pancreas, and brain cancer, notably not by damaging genetic material directly or indirectly, but by activating cellular signalling cascades at the wrong time and/or place in the body.

Various devices, systems and apparatuses as well as corresponding sensors to detect constituents of a surrounding medium have been developed over the past years. The detection of constituents in a medium or surrounding medium can also be referred to as environmental monitoring. Generally, environmental monitoring refers to the characterization of a condition or quality of a surrounding medium or environment over time and may, for example, be conducted to ensure that the chemical composition of the environment or surrounding medium does not pose a risk for human or animal health.

The detection of constituents in a surrounding medium or environmental monitoring can be an important tool employed for identifying and quantifying impacts of human activities on the natural environment, commonly soil, water, air or ecosystems, for example, for estimating a risk attributed to such impacts and for developing strategies for their reduction or elimination. Environmental monitoring can be conducted on a global level, for instance monitoring climate change or the microplastic contamination of the seas, on a regional level, for instance monitoring pollution of a particular river, or on a local level, for instance monitoring air quality at an industrial facility or the water quality in a fish farm. Local environmental monitoring can often be conducted to assure compliance of an industrial or agricultural facility with regulatory guidelines. It ultimately aims at maintaining the safety of employees, the nearby population and the environment by detecting and eliminating emissions, thereby reducing exposure and hence the associated risk. Such monitoring can be conducted in a targeted manner, as commonly the nature of the emission to be expected is well known. For example, in close proximity to fish farms, antibiotic residues in the water may be monitored or detected based on environmental monitoring. In agricultural areas, pesticide residues in ground- and/or river water may be of interest and near oil refineries, the concentration of volatile hydrocarbons in the surrounding air may be monitored and used as a marker for leakage of products at any stage of the refining process.

Environmental monitoring or the detection of constituents of a surrounding medium in general, however, may also be utilized in individual or personal applications, such as for monitoring a private or personal environment of a human being. For instance, the concentration of dust, pollen, and/or fungal spores, volatile organic carbon (VOC), nitric oxides (NOₓ) or UV irradiation may be measured in the air in close proximity to a person or individual having a corresponding detection device at home or carrying it, for example, in the car or attached to a baggage item, such as a backpack or handbag. The objective of such applications may be to reduce exposure of the individual, usually by escaping the exposure rather than by eliminating the source, because the latter may be difficult to achieve, as is for example the case with UV, pollen or ozone exposure. In particular in such individual or personal applications of environmental monitoring, it may be challenging that an unknown or undefined source of pollution is to be detected. Obtaining a comprehensive estimate of, for example, a current and local quality of the environment or surrounding medium may require monitoring various environmental parameters simultaneously, such as for instance particulate matter, VOC, NOₓ, ozone, UV, electrosmog and ionizing radiation.

Various devices and corresponding sensors have been developed for this purpose over the past years. Some of these devices and sensors can monitor a plurality of environmental parameters simultaneously and can allow for a real-time tracking of air quality, optionally including a location- and time-specific prediction of air pollution, for example for optimal route planning. These known or conventional devices for environmental monitoring, however, are usually limited in that the detection modes or sensors utilized are either highly selective or largely non-selective. For example, known exogenous noxae, such as NOₓ, carbon monoxide or ozone, can be selectively detected using highly sensitive and highly selective electrochemical or spectrometric sensors. Whilst such highly selective sensors may give valuable information on the concentration of a particular molecule, agent or compound to be detected in the surrounding medium, other molecules, agents or compounds that may potentially be harmful may not be detected, particularly if the respective molecules, agents or compounds are not known. On the other hand, a common type of non-selective sensors utilized in environmental monitoring are VOC sensors, which rely on, for instance, photoionization and detect any organic molecule that is ionized by light of a given frequency or energy. The ionization potential of an organic molecule may, however, not be related to its toxicity or bioactivity. By way of example, pinene, which is a plant derived terpene responsible for the characteristic smell of pine trees, and 1.1-dimethylhydrazine, which is a rocket propellant, have substantially identical ionization potentials of 8.07 eV and 8.05 eV, respectively, and are both ionized by UV light of sufficiently high frequency or energy of about 8 eV. Their acute toxicity or bioactivity, however, differs significantly. On the other hand, acetaldehyde, a carbonyl compound frequently used in synthetic chemistry has an ionization potential of 10.2 eV, and hence will not be detected by a VOC sensor operating at about 8 eV, but has significant acute toxicity or bioactivity.

As is evident from the aforementioned examples, devices employing highly selective sensors may fail to detect various compounds, agents or molecules, which are not detectable by the sensors, but may potentially adversely affect human health. Accordingly, the number of false negatives may be rather high when utilizing highly selective sensors. Devices employing non-selective sensors, on the other hand, may provide readings that are of limited health relevance, this is, the number of false positives may be high. In addition, devices with non-selective sensors may also fail in detecting other compounds, molecules or agents that may have adverse effects towards human health, this is, the number of false negatives may also be high. In other words, devices with non-selective sensors may detect broad groups of chemical compounds, molecules or agents having certain physicochemical properties in common, but such devices may not be able to distinguish between molecules, compounds or agents that potentially bear a health risk from those that do not.

It may, therefore, be desirable to provide for an improved device, system, apparatus, and method for detecting one or more bioactive agents in a surrounding medium, for example reporting a reduced number of false positives and/or false negatives.

This is achieved by the subject-matter of the independent claims. Optional features are provided by the dependent claims and by the following description.

Aspects of the present disclosure relate to a sensor chip, a sensing device, a sensing system, use of one or more of the aforementioned, and a method of detecting one or more bioactive agents in a surrounding medium. Any disclosure presented hereinabove and hereinbelow with reference to one aspect of the present disclosure, equally applies to any other aspect of the present disclosure.

According to an aspect of the present disclosure, there is provided a sensor chip operatively couplable to a sensing device for detecting one or more bioactive agents in a surrounding medium and/or a medium surrounding the sensor chip. The sensor chip comprises a reaction cell containing a plurality of receptor protein complexes, and a membrane separating the reaction cell from the surrounding medium and being permeable for the one or more bioactive agents. Therein, the receptor protein complexes are configured to bind to the one or more bioactive agents in the reaction cell, such that a detectable state change of at least a part of the receptor protein complexes is induced.

As will also be further elucidated hereinbelow, utilizing receptor protein complexes in the reaction cell that can bind to bioactive agents entering the reaction cell from the surrounding medium can allow to leverage the advanced and sophisticated ability of living organisms to detect bioactive agents in the medium surrounding them. As a consequence, a number of false positive events and false negative events detected or reported by the sensor chip, the sensing device and/or the sensing system of the present disclosure can be significantly reduced, thereby allowing for a comprehensive, reliable, and accurate detection of bioactive agents in the surrounding medium.

The reaction cell of the sensor chip can refer to or denote a compartment or chamber containing or comprising the receptor protein complexes, wherein at least a portion, part or region of the reaction cell may be separated from the surrounding medium by the membrane. The reaction cell can have any appropriate size, shape, geometry, form or volume.

The membrane may generally be permeable for the one or more bioactive agents to be detected by means of the sensor chip. For instance, the membrane may allow for a mass exchange between the reaction cell and the surrounding medium. In particular, the membrane may be arranged and configured, such that the one or more bioactive agents can enter the reaction cell from the surrounding medium through the membrane, for example based on diffusion.

As used herein, a bioactive agent may refer to a molecule, agent or compound able to trigger a physiological response in a living organism, such as a human or animal, for example upon binding to a receptor protein of the living organism equivalent or corresponding to the receptor protein complexes of the sensor chip. Therein, triggering a physiological response can include triggering a cellular defence mechanism or cellular response in the living organism. Alternatively or additionally, triggering a physiological response can include one or more of activating, inhibiting, and eliminating a biochemical process in the living organism.

For instance, a bioactive agent taken-up by a living organism and binding to a hormone receptor protein of the living organism, can activate cellular responses in the absence of the activity of endogenous signalling, as discussed hereinabove. As bioactive agents trigger physiological responses in living organisms, they are usually also related to or associated with a certain toxicity or health risk for the living organism. Accordingly, by detecting the one or more bioactive agents with the sensor chip of the present disclosure, a toxicity or pollution of the surrounding medium can be reliably determined or assessed.

The receptor protein complexes, as used herein, may generally refer to or denote functional complexes configured to bind to one or more bioactive agents and change their state upon binding, which can be detected or measured by means of the sensor chip. Therein, each receptor protein complex may comprise at least one ligand binding domain configured to bind to at least one bioactive agent, which can also be referred to as ligand in this context. For example, each receptor protein complex may comprise at least one ligand binding domain of a receptor protein that can be found in a living organism in similar, identical or equivalent structure, sequence or form.

The state change induced by binding of one of the receptor protein complexes of the sensor chip to at least one bioactive agent may, therefore, reflect, resemble or be indicative of the cellular or physiological response triggered when the bioactive agent binds to an equivalent or similar receptor protein in a living organism. Accordingly, when a state change is detected or determined by means of the sensor chip, it can be assumed that the bioactive agent would trigger a physiological response in a living organism. The sensor chip according to the present disclosure, therefore, can provide a significant advantage over sensors that non-selectively respond to a broad range of chemical compounds based on certain structural properties, which may not be related to the compounds' bioactivity, as is the case for example with VOC sensors. Further, it is noted that the chemical structure of the bioactive agent that binds to one of the receptor protein complexes as well as its source may be unknown. The sensor chip according to the present disclosure, therefore, can also provide a significant advantage over sensors that selectively and exclusively detect known bioactive agents, for example noxae.

Binding of a receptor protein complex can result in, be associated with or be accompanied by different types of state changes of the receptor protein complexes, including changes in conformation, structure, shape, position, localization, composition, chemical reactivity, chemical activity, and others. Alternatively or additionally, a state change induced in a receptor protein complex may result in a modification or alteration of at least a part of the reaction cell and/or the membrane. Such different state changes of the receptor protein complexes and/or the modifications or alterations of at least a component of the sensor chip associated therewith may be detected by different approaches, for example using detection principles or modes. As will be discussed in detail hereinbelow, all these different detection principles are envisaged in the context of the present disclosure.

In an example, the state change of the at least part of receptor protein complexes may be associated with a change in a conformational state of the at least part of receptor protein complexes. For example, the receptor protein complexes may be in their native state in the reaction cell and may be configured to change their conformation upon binding to one of the bioactive agents.

Alternatively or additionally, the state change of the receptor complexes may be associated with a change in a localization and/or position of the at least part of the receptor protein complexes within the reaction cell. Accordingly, the receptor protein complexes may be configured to change a position and/or localization within the reaction cell upon binding to the one or more bioactive agents. The change in position may be associated with or include a movement of the respective protein complex or at least a part thereof within the reaction cell. Such change in position and/or localization may, for example, be induced by a change in conformation of the receptor protein complex or another ligand binding to the receptor protein complex.

Alternatively or additionally, the state change may be associated with a change in a composition of the at least part of the receptor protein complexes. For instance, composition of a receptor protein complex may be changed upon binding to a bioactive agent based on dissociation or exchange of components or moieties of the receptor protein complex, or by binding of the receptor protein complex to another component or moiety. Accordingly, the receptor protein complexes may be configured to dissociate into one or more components upon binding to the one or more bioactive agents, may be configured to exchange one or more components upon binding to the one or more bioactive agents, and/or may be configured to bind to one or more moieties upon binding to the one or more bioactive agents.

Optionally, a change in composition of the at least part of the receptor protein complexes change may be associated with a change in a mass of the at least a part of the receptor protein complexes, for example if one or more moieties are released upon binding to the bioactive agent. Alternatively or additionally, a change in a mass of at least a part of the reaction cell and/or the membrane may be associated with the induced state change and may be detected by means of the sensor chip.

Further, the induced state change may be associated with one or more of a change in a physical property of the at least a part of the receptor protein complexes, a change in a physical property of at least a part of the reaction cell and/or the membrane, a change in an optical property of at least a part of the reaction cell and/or the membrane, a change in a chemical property of the at least a part of the receptor protein complexes, a change in a chemical property of a substrate contained in the reaction cell, a change in a conductivity of a substrate contained in the reaction cell, and a change in a concentration of free fluorescent or light absorbing molecules within at least a part of the reaction cell.

Any one or more of the aforementioned state changes and/or modifications or alterations of at least a part or component of the sensor chip associated therewith can be utilized to reliably detect presence of the one or more bioactive agents in the surrounding medium. Accordingly, the detectable one or more state changes of the at least part of the receptor protein complexes may be indicative of a presence of the one or more bioactive agents in the surrounding medium.

In an example, at least a part of the receptor protein complexes may be confined to or immobilized at at least one functional surface of the reaction cell, wherein the at least part of the receptor protein complexes may be configured to dissociate from the at least one functional surface upon binding to the one or more bioactive agents. In other words, the at least part of the receptor protein complexes confined to or immobilized at the at least one functional surface may be released or liberated from the at least one functional surface upon binding to one or more bioactive agents. The dissociation from the at least one functional surface may, for example, result in or be associated with one or more of a change of the mass of the at least one functional surface, the membrane or another inner surface of the reaction cell, a change in an optical property of the at least one functional surface, the membrane or another inner surface or the substrate contained in the reaction cell, and a change of another property or characteristic of one or more of the functional surface, the membrane and the reaction cell. Any one or more of such changes can be detected by means of the sensor chip and allow for an accurate detection of the one or more bioactive agents.

By way of example, at least a part of the receptor protein complexes may be covalently linked to at least one functional surface of the reaction cell. Accordingly, at least a part of the receptor protein complexes may be confined to or immobilized at the at least one functional surface based on covalently linking the receptor protein complexes to the at least one functional surface. For instance, at least a part of the receptor protein complexes may be confined to or immobilized at at least one functional surface of the reaction cell by a ligand, for example a low affinity ligand or a high affinity ligand, covalently linked to the at least one functional surface of the reaction cell. Therein, at least a part of the at least one functional surface may comprise or be coated with said ligands. While both low and high affinity ligands may be used, low affinity ligands may provide a stronger signal when compared to high affinity ligands.

Alternatively or additionally, the receptor protein complexes may be electrostatically confined to the at least one functional surface of the reaction cell. This can include, for example, ionic binding or binding by weak interactions, such as van der Waals forces. Electrostatic confinement can be provided passively, for instance based on a charge carried by the at least one functional surface, or actively, for instance based on charging one or more electrodes of the sensor chip at or near the at least one functional surface.

The at least one functional surface of the reaction cell may be defined by an inner surface of the membrane facing the reaction cell. In other words, an inner surface of the membrane, which faces or is directed towards an interior of the reaction cell, may constitute the at least one functional surface.

Alternatively or additionally, the at least one functional surface may include an inner surface of the reaction cell, which inner surface is disposed towards the membrane. The inner surface being disposed towards the membrane may mean that a surface normal vector of the inner surface and a surface normal vector of the membrane are directed transverse to each other. For example, the at least one functional surface may include an inner surface of the reaction cell arranged opposite to the membrane or next to the membrane.

In an example, the surrounding medium may include environmental air, atmospheric air or air in an environment or surrounding of the sensor chip. Accordingly, the sensor chip of the present disclosure may be configured to detect one or more bioactive agents in environmental air. Hence, the sensor chip may be utilized, for example, for environmental monitoring and/or monitoring of a quality of environmental air.

Alternatively, the surrounding medium may include water. Accordingly, the sensor chip of the present disclosure may be configured to detect one or more bioactive agents in water. Hence, the sensor chip may be utilized, for example, for environmental monitoring and/or monitoring of a quality of water, such as surface water, ground water, sea water, water of a river, water at a fish farm, pool water, or the like. It is noted that the sensor chip according to the present disclosure can also be utilized to advantage to detect one or more bioactive agents in other media or substances, such as soil.

The membrane may include an outer surface configured to contact the surrounding medium and may include an inner surface facing the reaction cell. Accordingly, the outer surface of the membrane may be directed towards the surrounding medium and the inner surface of the membrane may be directed towards an interior of the reaction cell. At least during use of the sensor chip, the outer surface of the membrane may be configured to continuously contact the surrounding medium, which can allow to continuously monitor the surrounding medium in terms of presence of one or more bioactive agents.

The reaction cell may, in an example, contain one or more of a liquid, gelatinous and semisolid substrate. Therein, the reaction cell may be partly or entirely filled with the substrate. For instance, the substrate may be composed such that the receptor protein complexes maintain a native state, particularly when not bound to one or more bioactive agents. Providing such a liquid, semisolid and/or gelatinous substrate in the reaction cell can significantly increase a lifetime of the receptor protein complexes and hence a lifetime of the sensor chip.

By way of example, the substrate may comprise at least one of an aqueous solution, an isotonic solution, and a buffered solution. Accordingly, the reaction cell may contain an aqueous, isotonic, buffered liquid, gelatinous and/or semisolid substrate, for example a gelatinous matrix, in which the receptor protein complexes and optional additional proteins that might be present in the reaction cell maintain their native state.

Optionally, the substrate may comprise a stabilizing protein for stabilizing the receptor protein complexes, for instance in their native state. Alternatively or additionally, the substrate may comprises a surface-active molecule or molecule complex, such as for example one or more of tween or triton X-100.

The membrane of the sensor chip may be permeable for gases. Permeability of the membrane to gases may be of particular advantage if the surrounding medium contains or comprises air, which can traverse the membrane and enter the reaction cell through the membrane. Alternatively or additionally, the membrane may be impermeable for water or aqueous liquid. Impermeability of the membrane to water or aqueous liquid may prevent leakage of substrate or other liquid out of the reaction cell, thereby ensuring functionality of the sensor chip and increasing its lifetime.

In an example, the membrane can include a plurality of pores, preferably filled with gas to exclude water. Such configuration of the membrane can efficiently ensure permeability to gases as well impermeability to water or aqueous liquid over an extended lifetime of the sensor chip.

For instance, the membrane may include at least one of porous carbon paper material and perforated fluoropolymer. It is noted, though, that the membrane can also include other materials or polymers, including lipids.

In a further example, the sensor chip may further comprise a grid covering at least a part of an outer surface of the membrane. The grid may be arranged and configured to protect the membrane and/or reaction cell from physical damage from the outside. The grid may be a rigid open grid. The grid may be integrally formed with a housing of the sensor chip or it may be formed as separate part or member attached to a housing of the sensor chip.

Alternatively or additionally, the sensor chip may further comprise a sealing cover covering at least a part of an outer surface of the membrane and configured to prevent contact of the membrane with the surrounding medium. The sealing cover may be in direct contact with the membrane or may be spaced apart from the membrane. For example, the sealing cover may be arranged at an outer surface of a grid covering at least a part of the outer surface of the membrane. The sealing cover may particularly be utilized to cover the membrane's outer surface and block bioactive agents from entering the reaction cell when the sensor chip is not in operation. This can increase the sensor chip's lifetime or lifespan, because the amount of receptor protein complexes in the reaction cell may decrease over time if not covered by the sealing cover.

Generally, the sealing cover may be single-use only or the sealing cover may be re-used to cover the membrane. Accordingly, the reaction cell may be sealable and/or re-sealable by covering at least a part of an outer surface of the membrane with the sealing cover.

In an example, the sealing cover may include an adhesive film, for example allowing to detachably attach or affix the sealing cover to the sensor chip. For example, an adhesive film may be arranged at at least a part of a surface of the sealing cover, such as at a perimeter of the surface, which can allow to securely attach the sealing cover to the sensor chip and ensure that the membrane or reaction cell is comprehensively sealed by the sealing cover against the surrounding medium. It should be noted, however, that also other means of attaching the sealing cover to the sensor chip may utilized, including a snap-fit connection or other mechanical coupling, as well as a magnetic coupling.

The sealing cover may be air-tight and/or may be configured to block air from traversing the sealing cover. An air-tight configuration and attachment of the sealing cover to the sensor chip can ensure that no surrounding medium or no bioactive agents can enter the reaction cell, which could potentially decrease the number of receptor protein complexes in the reaction cell over time. As noted above, this sealing of the reaction cell can be of particular advantage when the sensor chip is not in operation, for example when storing the sensor chip.

The sensor chip may be configured in shape and size to be at least partly inserted into the sensing device. Accordingly, the sensor chip may be shaped and sized, such that it can be at least partly inserted into the sensing device. Such partial insertion can ensure correct positioning of the sensor chip in the sensing device, can ensure proper connection or coupling between the sensor chip and sensing device, and can protect the sensor chip from damage.

For example, the sensor chip can be configured in shape and size to be at least partly inserted into a socket of the sensing device. In other words, the sensing device can comprise at least one socket for at least partly receiving the sensor chip. The socket may, for example, be slot-like formed and the sensor chip can be inserted into the socket by pushing the sensor chip into the socket.

Optionally, the sensor chip may comprise at least one surface feature at an outer surface of a housing of the sensor chip, wherein the at least one surface feature of the sensor chip is formed complementary to at least one surface feature of the socket to ensure correct positioning of the sensor chip in the socket. Therein, the at least one surface feature of the sensor chip may optionally be configured to engage with the at least one surface feature of the socket to fix the sensor chip in the socket. Accordingly, the surface feature of the socket and the surface feature at the housing of the sensor chip may provide a click or snap-fit mechanism for fixing the sensor chip in the socket.

Alternatively or additionally, the sensor chip may include one or more magnets or magnetic elements for magnetically coupling the sensor chip to the sensing device. Also other means of fixing the sensor chip to the sensing device may be utilized, such as a screw-locking or other mechanical locking.

As noted above, one or more different detection principles or modes may be applied to detect the state change of the at least part of the receptor protein complexes induced by binding to one or more bioactive agents. For example, the state change of the at least part of the receptor protein complexes may be detectable based on an optical measurement. Such optical measurement may be performed within at least a part of the reaction cell, at at least a part of the membrane or outside of the reaction cell. Alternatively or additionally, the state change may be detectable based on detecting fluorescent light and based on fluorescent excitation of one or more components of the receptor protein complexes. For example, the receptor protein complexes may include at least one fluorescent label and may be configured to dissociate upon binding to one or more bioactive agents, thereby releasing the at least one fluorescent label into the reaction cell. Fluorescence light emitted by fluorescent labels released from the receptor protein complexes upon binding may be detected in order to detect the state change. Alternatively or additionally, the state change may be detectable based on light scattering, for example by passing light through at least a part of the reaction cell and by measuring a change in intensity of the light passing through the reaction cell. Alternatively or additionally, the state change may be detectable based on determining one or more optical properties of at least one functional surface of the reaction cell. Alternatively or additionally, absorption of electromagnetic radiation within the reaction cell or at the at least one functional surface may be utilized to detect the state change of the receptor protein complexes. For instance, binding of receptor protein complexes to one or more bioactive agents may lead to dissociation of one or more components of the receptor protein complexes and/or dissociation of the receptor protein complexes from the at least one functional surface, which can result in a detectable change of the optical properties of the at least one functional surface and/or the substrate contained in the reaction cell. Alternatively or additionally, the state change may be detected based on determining a conductivity and/or a change in conductivity of a substrate contained in the reaction cell. For this purpose, the sensor chip may comprise one or more electrodes arranged in an interior of the reaction cell. Alternatively or additionally, the state change may be detectable based on detecting an electrochemical process occurring in the reaction cell upon binding of the at least part of the receptor protein complexes to one or more bioactive agents. Alternatively or additionally, the state change may be detectable based on determining a mass and/or a change in mass of at least one functional surface of the reaction cell, for example based on determining a mass and/or change in mass of receptor protein complexes confined to or immobilized at the at least one functional surface of the reaction cell. For instance, the receptor protein complexes may be confined at the at least one functional surface and may dissociate therefrom upon binding to the one or more bioactive agents, which can result in a measurable change in mass of the at least one functional surface or the membrane. Alternatively or additionally, a change of one or more physical or optical properties of the at least one functional surface may be detectable based on surface plasmon resonance at the at least one functional surface of the reaction cell. It is emphasized that any one or more of the aforementioned detection principles or even other detection principles may be utilized to detect the state change induced by binding of the receptor protein complexes to one or more bioactive agents.

It is noted that further technical means for actually detecting one or more of the aforementioned state changes, such as corresponding sensors or sensing elements, may be included in the sensor chip and/or the sensing device. Accordingly, one or both the sensor chip and the sensing device may be configured to determine the state change of the at least part of the receptor protein complexes based on an optical measurement, based on detecting fluorescent light, based on fluorescent excitation of one or more components of the receptor protein complexes, based on light scattering, based on determining a conductivity of a substrate contained in the reaction cell of the sensor chip, based on a electrochemical process occurring in the reaction cell, based on determining one or more optical properties of at least one functional surface of the reaction cell, based on determining absorption of electromagnetic radiation, based on determining a mass of at least one functional surface of the reaction cell, based on determining a mass of receptor protein complexes confined to or immobilized at at least one functional surface of the reaction cell, and based on surface plasmon resonance at at least one functional surface of the reaction cell.

According to an example, the sensor chip includes one or more connectors for operatively coupling the sensor chip to the sensing device. Such operative coupling may mean that the sensor chip may be connected via the one or more connectors to the sensing device, such that the state change of the at least part of the receptor protein complexes upon binding to the one or more bioactive agents is detectable.

In an example, the one or more connectors may include at least one optical connector configured to couple electromagnetic radiation from the sensing device into the reaction cell for optically detecting the change of conformational state of the at least part of the receptor protein complexes upon binding to the one or more bioactive agents. Alternatively or additionally, the one or more connectors may include at least one further optical connector for coupling electromagnetic radiation out of the reaction cell. For instance, the sensor chip may comprise a first optical connector for transmitting electromagnetic radiation from the sensing device into the sensor chip and a second optical connector for transmitting electromagnetic radiation out of the sensor chip into the sensing device. The sensor chip may also include more than two optical connectors for optically coupling the sensor chip to the sensing device.

The at least one optical connector and/or the at least one further optical connector, for example the first and second optical connectors, may include at least one aperture arranged in a housing of the sensor chip. Via the at least one aperture of the corresponding optical connector, electromagnetic radiation may be coupled into and/or out of the sensor chip in order to optically detect the state change of the receptor protein complexes upon binding to the one or more bioactive agents.

Optionally, the at least one aperture may be sealed with a layer of material translucent to electromagnetic radiation of a predefined wavelength. For example, the state change may be detected based on fluorescent excitation of one or more fluorescent labels of the receptor protein complexes, which may be released upon binding to a bioactive agent. Therein, the layer of material sealing the aperture may be permeable or translucent to at least fluorescent excitation light of a predefined wavelength or range of wavelengths, allowing to couple the fluorescent excitation light into the reaction cell. Alternatively or additionally, the layer of material sealing the aperture may be permeable or translucent to at least the fluorescent light emitted by the fluorescent labels.

The at least one aperture may, for example, be shaped, oriented or formed, such light or electromagnetic radiation entering the reaction cell through the aperture results in a sheath of light traversing at least a part of the reaction cell. For instance, the at least one aperture may be oriented parallel to a longitudinal axis of the reaction cell.

In yet another example, the sensor chip further includes at least one optical guide for guiding electromagnetic radiation through the reaction cell for optically detecting the state change of the at least part of the receptor protein complexes upon binding to the one or more bioactive agents. Therein, the at least one optical guide may traverse at least a part of the reaction cell. For example, the at least one optical guide may extend parallel to a longitudinal axis of the reaction cell through the reaction cell. Arranging one or more optical guides within the reaction cell may generally allow for a high-sensitivity optical measurement, thereby allowing to accurately and reliable detect the state changes of the receptor protein complexes upon binding.

The at least one optical guide may be aligned with and optically coupled to at least one optical connector of the sensor chip. Accordingly, electromagnetic radiation or light may be coupled into and/or out of the at least one optical guide via the at least one optical connector.

By way of example, the sensor chip may comprise at least two optical guides and at least one reflexive element arranged at an end of the at least two optical guides, wherein the at least one reflexive element optically couples the at least two optical guides. In other words, electromagnetic radiation may be passed through one of the optical guides, reflected at the at least one reflexive element and coupled into the further optical guide. Optionally, each of the at least two optical guides may be optically coupled with at least one optical connector of the sensor deceive arranged at an end of the respective optical guide opposite to the at least one reflexive element. In such configuration, a pathlength of electromagnetic radiation traversing the reaction cell through the at least two optical guides may be maximized, which can increase sensitivity of the sensor chip.

In yet another example, the one or more connectors of the sensor chip may include at least one electrical connector for electrically coupling the sensor chip to the sensing device. Such electrical connector may allow electrical signals, for example control signals or detection signals, to be provided by the sensing device to the sensor chip or vice versa. Alternatively or additionally, the at least one electrical connector may allow for a data communication or communicative coupling between the sensor chip and the sensing device.

In an example, the sensor chip further includes one or more electrodes at least partly arranged within the reaction cell and configured to determine a conductivity or change in conductivity of a substrate or composition within the reaction cell. As mentioned hereinabove, the state change induced upon binding of the at least part of the receptor protein complexes may include dissociation of the receptor protein complexes into several components and/or dissociation of the receptor protein complexes from the at least one functional surface. Any of these state changes may lead to a change in conductivity of the substrate contained in the reaction cell, which can be measurable by means of the one or more electrodes with high accuracy, sensitivity and precision.

In yet another example, the sensor chip may further include at least one detection window translucent for electromagnetic radiation emitted and/or scattered by at least one or more components of the receptor protein complexes. For instance, the at least one detection window may be translucent for fluorescent light emitted by the at least one or more components of the receptor protein complexes, such as for example one or more fluorescent labels released upon binding to one or more bioactive agents.

Alternatively or additionally, the at least one detection window may be opaque for fluorescence excitation light. Hence, by means of the detection window fluorescent excitation light may be blocked from exiting the reaction cell. Such configuration may allow to increase sensitivity of the sensor chip, in particular in case an intensity of fluorescent excitation light is much higher than an intensity of the actual fluorescent light emitted by the fluorescent labels.

The at least one detection window may, for example, be arranged opposite to, disposed towards, and/or oriented towards the membrane. Such arrangement of the detection window can ensure that changes of an optical property of the membrane or a functional surface of the reaction cell can be reliably detected through the detection window of the sensor chip.

In an example, an inner surface of the at least one detection window facing the reaction cell may be at least partly coated with molecular trapping complexes configured to bind to at least a component of the receptor protein complexes, such that receptor protein complexes bound to the one or more bioactive agents are trapped at the inner surface. The inner surface of the at least one detection window may, thus, act as a sink for receptor protein complexes within the reaction cell, which have already bound to one or more bioactive agents. By means of the coated inner surface a sensitivity of the sensor chip can be kept at a high level even during long periods of operation in a surrounding medium containing a significant amount of bioactive agents. Such configuration may be of particular advantage when detecting the state change optically, for instance based on fluorescent excitation. For example, after prolonged operation or high exposure of the sensor chip to bioactive agents, a large fraction of the receptor proteins complexes may have been bound to bioactive agents, dissociated and/or liberated from the membrane, which may render a proper detection of additional fluorescence induced by newly bound receptor protein complexes inaccurate. Hence, by means of the coated inner surface of the at least one detection window, it can be ensured that already bound receptor protein complexes or components thereof, such as one or more fluorescent labels, are trapped at the coated inner surface and cannot affect or influence the optical measurement.

To actually bind the receptor protein complexes or one or more components thereof to the molecular trapping complexes, the receptor protein complexes may comprise an affinity tag, preferably binding with high affinity to the molecular trapping complexes. The affinity tag may, for instance, be biotin, in which case the molecular trapping complexes may comprise streptavidine. Alternatively or additionally, the affinity tag may be a histidine tag, in which case the molecular trapping complexes may comprise chelated nickel ions.

Alternatively or additionally, the inner surface of the at least one detection window facing the reaction cell may at least partly be coated with quenching molecules for quenching scattered light and/or fluorescent light emitted by at least one component of the receptor protein complexes, such as a fluorescent label. Alternatively or additionally, an inner surface of the membrane facing the reaction cell may be at least partly coated with quenching molecules for quenching scattered light and/or fluorescent light emitted by at least a component of the receptor protein complexes. Quenching of the scattered light and/or fluorescent light can decrease or even eliminate noise originating from light scattering or from fluorescence emission not attributed to receptor protein complexes binding to one or more bioactive agents.

In an example, the sensor chip and/or the reaction cell may have an elongated shape. Such elongated shape may allow to increase a surface of the membrane and/or reaction cell exposed to the surrounding medium, which can allow to increase a quality and precision of the actual detection of the bioactive agents. Therein, the membrane may optionally be arranged at a longitudinal side of the sensor chip.

In yet another example, the reaction cell may have a rectangular cross-section. Alternatively or additionally, the reaction cell may be formed as parallelepiped. It should be noted, however, that also other geometries, shapes or forms of the reaction cell are envisaged in the context of the present disclosure.

For instance, the reaction cell may have a circular, elliptical or oval cross-section and/or may be tubular shaped. Therein, the membrane may at least partly encompass the reaction cell along a perimeter or outer circumference of the reaction cell. Also such configuration may allow to increase or maximise the surface of the membrane through which bioactive agents can enter the reaction cell, thereby allowing to increase accuracy and precision of the detection of bioactive agents.

In a further example, the sensor chip may further include at least one reservoir fluidly couplable with the reaction cell, wherein the at least one reservoir is configured to supply de-ionized water to the reaction cell. Accordingly, the at least one reservoir may be at least partly filled with de-ionized water. Th reservoir may also be referred to as water reservoir herein. By means of the reservoir, substrate, or liquid escaping from the reaction cell over time, for example water passing through the membrane, may be compensated for by supplying de-ionized water to the reaction cell.

Alternatively or additionally, the reaction cell may be in a dry state or not contain a liquid, semisolid and/or gelatinous substrate before using the sensor chip for the first time, and the sensor chip may be activated by supplying de-ionized water from the reservoir into the reaction cell.

In an example, at least a part of a wall of the at least one reservoir may be displaceable, flexible or movable, such that a volume of the reservoir may be adjustable. Such configuration can allow to dynamically compensate for any loss or leakage of substrate, liquid, fluid or water from the reaction cell over time.

For instance, at least a part of the at least one reservoir may be formed as flexible bag or flexible blister. Therein, a volume of the reservoir may be adjusted in accordance with an amount or volume of de-ionized water supplied to the reaction cell, in particular without posing significant resistance to deflation if water is leaving the reservoir towards the reaction cell.

Optionally, the at least one reservoir may be encompassed by a portion of a housing of the sensor chip, which comprises at least one opening for pressure equalization. Via the at least one opening, surrounding medium, for example air, can enter the housing when water is pulled from the reservoir towards the reaction cell, for example by a higher osmolarity in the reaction cell compared to the reservoir.

Alternatively or additionally, the sensor chip may further include at least one movable piston configured to adjust a volume of the at least one reservoir. Also this configuration can allow to dynamically compensate for any loss or leakage of substrate, liquid, fluid or water from the reaction cell over time.

Optionally, the at least one reservoir may be fluidly coupled or connected to the reaction cell by a semi-permeable membrane blocking salts to diffuse from the reaction cell into the reservoir. The semi-permeable membrane may ensure that the salts remain in the reaction cell and maintain a gradient in osmolarity leading to a flow of de-ionized water from the reservoir into the reaction cell.

In yet another example, the sensor chip may further include at least one blocking element configured to block fluid communication between the reaction cell and the at least one reservoir. For instance, the sensor chip may be activatable based on unblocking fluid communication between the reaction cell and the at least one reservoir using the at least one blocking element. In other words, the sensor chip may be activated by unblocking the fluid communication and by supplying de-ionized water from the reservoir into the reaction cell.

The at least one blocking element may, for example, comprise a water-impermeable membrane arranged between the at least one reservoir and the reaction cell. Therein, the sensor chip may be activatable based on or by disrupting at least a part of the water-impermeable membrane.

Alternatively or additionally, the at least one blocking element may comprises a movable pin operable to block or unblock fluid communication between the reaction cell and the at least one reservoir. Therein, the sensor chip may be activatable based on displacing the movable pin, such that fluid communication between the reaction cell and the at least one reservoir is unblocked or established.

According to an example, the receptor protein complexes may each comprise at least one ligand binding domain of a receptor protein configured to bind to the one or more bioactive agents and configured to change conformation upon binding to one of the bioactive agents. Optionally, at least some of the receptor protein complexes may comprise a plurality of ligand binding domains of one or more receptor proteins. Accordingly, a plurality of bioactive agents may be bound by a single receptor protein complex, which can increase overall sensitivity of the sensor chip.

Further optionally, the plurality of ligand binding domains may be of different type and may be configured to bind to different types of bioactive agents. In other words, the receptor protein complexes may comprise at least a part of, for example a ligand binding domain of, different types of receptor proteins configured to bind to different types of bioactive agents. Hence, a plurality of different types of bioactive agents may be detected with a single receptor protein complex.

According to an example, at least some of the receptor protein complexes may comprise at least one ligand binding domain of a receptor protein, which is a xenosensor protein or a hormone receptor protein. Alternatively or additionally, the receptor protein complexes may each comprise at least one ligand binding domain of a receptor protein selected from the group consisting of Aryl Hydrocarbon Receptor, Constitutive Androstane Receptor, Pregnane X Receptor, and Estrogen Receptor.

Alternatively or additionally, at least some of the receptor protein complexes may comprise at least one binding domain of an aryl hydrocarbon receptor or aryl hydrocarbon receptor protein. For instance, at least some of the receptor protein complexes may comprise at least the PAS (Per-ARNT-Sim) domain or the entire aryl hydrocarbon receptor protein, which may allow for detection of materials, compounds, molecules and/or agents, for example halogenated aromatic hydrocarbons, such as polychlorinated dibenzodioxins, dibenzofurans and biphenyls, and/or polycyclic aromatic hydrocarbons, such as 3-ethylcholanthrene, benzo[a]pyrene, benzanthracenes and benzoflavones.

Generally, at least a part of the receptor protein complexes may comprise a recombinant protein or a non-recombinant protein. Alternatively or additionally, the receptor protein complexes may each comprise at least a part of a monomer receptor protein, a homodimer receptor protein complex or a heterodimer receptor protein complex.

Further, at least some of the receptor protein complexes may comprise an organic or inorganic moiety dissociating from a remaining part of the respective receptor protein complex upon binding to the one or more bioactive agents.

By way of example, the receptor protein complexes may comprise at least one fluorescent label dissociating or being released from a remaining part of the respective receptor protein complex upon binding to the one or more bioactive agents. Using such receptor protein complexes, the state changes induced by binding of the receptor protein complexes to one or more bioactive agents can be detected based fluorescence excitation of the at least one fluorescent label dissociated or liberated from the remaining part of the receptor protein complexes. Optionally, at least some of the receptor protein complexes may comprise a plurality of fluorescent labels of same or different type. By way of example, the at least one fluorescent label may be a fluorescent dye or green fluorescent protein.

Alternatively or additionally, the receptor protein complexes may comprise a nanoparticle dissociating or being released from a remaining part of the respective receptor protein complex upon binding to the one or more bioactive agents. Using a nanoparticle, for example gold nanoparticle, as moiety that is released upon binding, a change in mass induced by the event of binding may be increased, which can increase sensitivity and precision of the detection of the bioactive agents.

In yet another example, the sensor chip further includes a chip identifier for identifying a type of the sensor chip and/or a type of receptor protein complex contained in the sensor chip. Similarly, the chip identifier may allow to identify the type of bioactive agents detectable with the sensor chip. In an example, the chip identifier may contain information or data indicative of the type of the sensor chip and/or the type of receptor protein complex contained in the sensor chip.

Optionally, the chip identifier may be readable by a user device and/or readable by the sensing device. As used herein, a user device may refer to any device operatively and/or communicatively couplable to the sensor chip or the sensing device, such as for example a smartphone, a tablet, a personal computer, a notebook, a smart device, a smart watch or any other computing device. Reading the chip identifier may in particular allow to provide information related to the type of sensor chip and/or the receptor protein complexes to the user device and/or the sensing device. This may further allow to ensure proper operation and control of the sensor chip by the sensing device, when the sensor chip is inserted into the sensing device.

In an example, the chip identifier may include one or more of a barcode, a QR code, an RFID tag, a label, and a data storage. The barcode, QR code, label or RFID tag may be read by the user device and/or the sensing device, based on optical detection or using an RFID reader employed in the user device and/or sensing device. Likewise, data stored on the data storage may be read by the user device and/or the sensing device based on establishing a data communication, for example via one or more connectors of the sensor chip.

In yet another example, the sensor chip may further include a data storage configured to store historic data indicative of a usage or remaining lifetime of the sensor chip. Also such data may be read by a user device and/or sensing device. Storing historic data on the data storage may in particular allow to re-use the sensor chip, for example by re-inserting it into a sensing device, while still ensuring proper functionality of the sensor chip, for example because the sensing device can determine that the lifetime of the sensor chip has not yet expired.

For instance, one or more operational parameters related to the use of the sensor chip may be measured throughout the life span or lifetime of the chip and stored on the data storage as historic data. In other words, the data storage may be configured to store one or more operational parameters indicative of the usage or remaining lifetime of the sensor chip.

Optionally, a notification may be provided by the sensing device and/or the user device if the remaining lifetime of the sensor chip reaches or falls below a predetermined threshold, for example due to depletion of the receptor protein complexes in the reaction cell, thereby indicating that the sensor chip should be replaced. Optionally, the sensing device and/or the user device may determine, based on the historic data, depletion of the receptor protein complexes in the reaction cell and determine the remaining lifetime of the sensor chip based on the determined depletion.

Alternatively or additionally, the sensing device and/or the user device may determine, based on the historic data, a cumulative exposure to bioactive agents, such as a cumulative air pollution. Optionally, such information can be provided to a user, for example at a user interface of the sensing device and/or the user device.

In an example, the data storage of the sensor chip may be accessible by the sensing device upon operatively coupling the sensor chip to the sensing device, for example upon at least partly inserting the sensor chip into the sensing device. Alternatively or additionally, the data storage of the sensor chip may be read by the sensing device upon operatively coupling the sensor chip to the sensing device. For example, the data storage and/or a chip identifier of the sensor chip may be automatically detected and/or read upon operatively coupling the sensor chip to the sensing device.

Generally, the sensor chip can include a plurality of reaction cells and/or membranes. Optionally, the plurality of reaction cells may comprise different types of receptor protein complexes configured to bind to different types of bioactive agents. These configurations may allow to increase sensitivity of the sensor chip and broaden the range of bioactive agents that can be detected by the sensor chip. Accordingly, a compact sensor chip, which can allow for a detection of multiple different bioactive agents with high precision can be provided.

In yet another example, the sensor chip further includes one or more sensors configured to determine the state change of the at least part of the receptor protein complexes. Optionally, the sensor chip may comprise processing circuitry configured to provide a detection signal indicative of a determined state change of the at least part of the receptor protein complexes. Accordingly, electronics and sensing elements for the actual detection of the state changes of the receptor protein complexes may be at least in part or entirely included in the sensor chip. Alternatively or additionally, however, one or more sensors and/or at least a part of the processing may be arranged in the sensing device.

For example, the sensor chip may comprise one or more optical sensors, such as photodetectors, for optically detecting the state change. Alternatively or additionally, the sensor chip may comprise one or more electrodes for detecting the state change based on a conductivity measurement within the reaction cell. Alternatively or additionally, the sensor chip may comprise one or more piezo elements arranged and configured for detecting the state change based on determining a mass and/or change in mass of receptor protein complexes immobilized at and/or released from at least one functional surface of the sensor chip. Alternatively, the one or more piezo elements may be arranged and configured for determining the mass and/or change in mass of receptor protein complexes immobilized at and/or released from at least one functional surface of the sensor chip based on determining a mass and/or change in mass of receptor protein complexes or one or more components thereof at one or more inner surfaces of the reaction cell disposed towards the at least one functional surface.

A further aspect of the present disclosure relates to a use of the sensor chip, as described hereinabove and hereinbelow, for detecting one or more bioactive agents. Any disclosure presented hereinabove and hereinbelow with reference to the sensor chip, equally applies to the use of the sensor chip.

In a further aspect of the of the disclosure, there is provided a sensing device for detecting one or more bioactive agents in a surrounding medium. The sensing device comprises a housing configured to at least partly receive at least one senor chip, as described hereinabove and hereinbelow, to operatively couple the sensing device to the at least one sensor chip. The sensing device further comprises processing circuitry configured to provide a detection signal indicative of presence of the one or more bioactive agents in the surrounding medium based on determining a state change of at least a part of the receptor protein complexes in a reaction cell of the at least one sensor chip, the state change being induced by binding of said one or more receptor protein complexes with one or more bioactive agents entering from the surrounding medium through a membrane of the sensor chip into the reaction cell of the sensor chip.

It is noted that the sensing device may optionally include one or more sensors to determine a state change of at least a part of the receptor protein complexes in the reaction cell of the at least one sensor chip. At least some of or all of the sensors may alternatively be included in the sensor chip. Similarly, at least a part of or the entire processing circuitry may be arranged or included in the sensor chip.

The processing circuitry may, for example, include one or more processors, one or more controllers or one or more micro-controllers for data processing or operational control of the sensing device and/or the sensor chip. Optionally, at least a part of the processing circuitry may be implemented on a printed circuit board. Alternatively or additionally, at least a part of the processing circuitry may be implemented as smart chip or smart device. Alternatively or additionally, at least a part of the processing circuitry may be implemented as application-specific integrated circuit, ASIC.

In yet a further aspect of the of the disclosure, there is provided a sensing device operatively couplable to at least one sensor chip, as described hereinabove and hereinbelow, for detecting one or more bioactive agents in a surrounding medium. The sensing device comprises at least one sensor configured to determine a state change of at least a part of the receptor protein complexes in a reaction cell of the at least one sensor chip, the state change being induced by binding of one or more receptor protein complexes with one or more bioactive agents entering from the surrounding medium through a membrane of the sensor chip into the reaction cell of the sensor chip. The sensing device further includes processing circuitry coupled with the at least one sensor, wherein the processing circuitry is configured to provide a detection signal indicative of presence of the one or more bioactive agents in the surrounding medium based on determining the state change of the at least part of the receptor protein complexes.

In an example, the surrounding medium may include environmental air. Alternatively or additionally, the sensing device may be or may be configured as an environmental monitoring device for monitoring a quality of environmental air.

The sensing device can be or can be configured as a stationary device and/or desktop device. The sensing device may, for example, be utilized at a home or office for monitoring a quality of the air in this environment. Alternatively, the sensing device may be at least temporally installed at other locations, such as in a car or the like. Generally, the sensing device may be configured to monitor the quality of the air continuously or in predefined time intervals.

Alternatively, the sensing device may be or may be configured as a portable device, a mobile device and/or a handheld device. In such configuration, the sensing device may be carried by a person to monitor the quality of the air surrounding the person, for example continuously or in predefined time intervals. The sensing device may be carried, for example, in a pocket, bag, backpack or handbag of the person, or may be attached to an item of the person, such as an item of cloths, a bag, backpack or handbag.

As used herein, the detection signal may refer to or denote an electronically processable signal, such as for example an electronic signal or a data signal. Optionally, the detection signal may be used by the processing circuitry to provide a reading indicative of the one or more detected bioactive agents, for example at a user interface of the sensing device. Alternatively or additionally, the detection signal may be transmitted from the sensing device to a user device and further processed by the user device to provide a reading indicative of the one or more detected bioactive agents.

The detection signal may be indicative of an amount of one or more bioactive agents per volume of surrounding medium, a mass of bioactive agent per volume of surrounding medium, a concentration of bioactive agent in the surrounding medium, a receptor protein activation potential of the surrounding medium, and a bioactivity of the surrounding medium. Accordingly, one or more of the aforementioned quantities may be determined by the processing circuitry and/or a user device based on processing the detection signal.

In an example, the sensing device may be mechanically couplable to the sensor chip. A mechanical coupling can include, for example, a snap-fit connection, at least partly engaging surface features of the sensor chip and the sensing device, mechanical fixation of the sensor chip at the sensing device, for instance by means of a screw-locking, or any other mechanical coupling. By mechanically coupling the sensor chip with the sensing device, it may be ensured that the sensor chip is in a predefined position and/or orientation to allow for an operational coupling between the sensing device and sensor chip. Accordingly, mechanically coupling the sensor chip and the sensing device may optionally include establishing an operational coupling between the sensor chip and the sensing device, which can for example include establishing one or more optical connections, one or more electrical connections, and/or one or more data connections. Hence, by mechanically coupling the sensor chip to the sensing device it may be ensured that the sensor chip is functional and operational.

Alternatively or additionally, the sensing device may be magnetically couplable to the sensor chip. For instance, the sensing device may comprise one or more magnets interacting with one or more magnetic counter parts or magnetic elements of the sensor chip for magnetically coupling the sensor chip to the sensing device. Also by magnetically coupling the sensor chip with the sensing device, it may be ensured that the sensor chip is in a predefined position and/or orientation to allow for an operational coupling between the sensing device and sensor chip. Accordingly, magnetically coupling the sensor chip and the sensing device may optionally include establishing an operational coupling between the sensor chip and the sensing device, which can for example include establishing one or more optical connections, one or more electrical connections, and/or one or more data connections. Hence, by magnetically coupling the sensor chip to the sensing device it may be ensured that the sensor chip is functional and operational.

In an example, the sensing device may be configured to at least partly receive the at least one sensor chip. For instance, the sensing device may comprise at least one socket configured to at least partly receive the at least one sensor chip. The sensor chip may be removably inserted at least partly into the sensing device or the at least one socket. Alternatively or additionally, the at least one socket may be configured to at least partly receive the at least one sensor chip, such that a housing of the sensing device at least partly encompasses the at least one sensor chip. At least partly inserting the sensor chip into the sensing device or socket may protect the sensor chip and may ensure that an operational coupling between the sensor chip and the sensing device can be established.

Optionally, the at least one socket may comprise at least one surface feature formed complementary to at least one surface feature of the sensor chip to ensure correct positioning of the sensor chip in the at least one socket. For example, the at least one surface feature of the at least one socket may include one or more guides for positioning the sensor chip within the socket. Alternatively or additionally, the at least one surface feature of the at least one socket may be configured to at least partly engage with the at least one surface feature of the sensor chip, such that the sensor chip is removably fixable in the socket. Accordingly, the surface feature of the socket and the surface feature at the housing of the sensor chip may optionally provide a click or snap-fit mechanism for fixing the sensor chip in the socket.

It is noted that the sensing device may comprise a plurality of sockets configured to receive a plurality of sensor chips of same or different type. For example, different types of sensor chips for detecting different types of bioactive agents or one or more environmental parameters, such as a UV radiation exposure or ozone exposure, may be utilized with a single sensing device. Hence, the sensing device can used to comprehensively monitor the environment.

The processing circuitry of the sensing device may be configured to determine presence of the one or more bioactive agents in the surrounding medium based on detecting, with at least one sensor of the sensor chip or the sensing device, a positional change of one or more receptor proteins within the reaction cell upon binding to one or more of the bioactive agents.

For example, the sensing device may be configured to determine one or more of a presence, a number, a mass, a density, and a mass density of receptor protein complexes dissolved in a substrate of the reaction cell of the at least one sensor chip. Alternatively or additionally, the sensing device may be configured to determine one or more of a presence, a number, a mass, a density, and a mass density of receptor protein complexes dissolved in a substrate of the reaction cell of the at least one sensor chip based on determining a change in at least one of an optical and an electrical signal transmitted to the sensor chip. Accordingly, the sensing device may be configured to detect the state change induced by binding of the at least part of the receptor protein complexes to one or more bioactive agents based on determining a change in at least one of an optical and an electrical signal transmitted to the sensor chip.

In an example, the receptor protein complexes may be configured to dissociate from at least one functional surface of the reaction cell upon binding to one of the bioactive agents, wherein the sensing device may be configured to determine one or more of a number, a mass, a density, and a mass density of receptor protein complexes immobilized at or liberated from at least one functional surface of the reaction cell.

Optionally, the sensing device may include one or more piezo elements configured to determine a mass and/or a change in mass of receptor protein complexes immobilized at or liberated from at least one functional surface of the reaction cell to detect the one or more bioactive agents in the surrounding medium. In an example, the one or more piezo elements of the sensing device may be arranged adjacent to, in close proximity to or even in contact with a component of the sensor chip carrying the at least one functional surface, such as the membrane or a part of a housing of the sensor chip disposed towards or arranged opposite to the membrane. The mass and/or change in mass may for example be determined based on exciting mechanical vibrations of the component of the sensor chip carrying the at least one functional surface, such as the membrane, and measuring characteristics of the mechanical vibrations, such as a damping behaviour. This may allow to determine, for example by the processing circuitry of the sensing device, the mechanical properties of the component of the sensor chip carrying the at least one functional surface and in turn may allow to determine the mass and/or change in mass of receptor protein complexes immobilized at or liberated from at least one functional surface of the reaction cell. It should be noted, however, that the one or more piezo elements may alternatively be arranged in the sensor chip and may be operationally controlled by the sensing device.

Alternatively or additionally, the one or more piezo elements may be configured to determine the mass and/or change in mass of receptor protein complexes immobilized at or liberated from the at least one functional surface of the reaction cell based on determining a mass of one or more components of the receptor protein complexes bound to an inner surface of the reaction cell disposed towards or arranged opposite to the at least one functional surface of the reaction cell. For instance, receptor protein complexes may be liberated from the at least one functional surface upon binding and diffuse towards the inner surface of the reaction cell disposed towards the at least one functional surface. Receptor protein complexes or components thereof may accumulate at the inner surface, which accumulation can be measured by determining the mass and/or change in mass of receptor protein complexes at the inner surface. Also in this configuration, the one or more piezo elements of the sensing device may be arranged adjacent to, in close proximity to or even in contact with a component of the sensor chip or reaction cell carrying the inner surface of the reaction cell. The mass and/or change in mass may for example be determined based on exciting mechanical vibrations of the component of the sensor chip, as described hereinabove.

In an example, the one or more piezo elements may be mechanically couplable to one or more functional surfaces of the reaction cell of the sensor chip. Alternatively or additionally, the one or more piezo elements may be mechanically couplable to one or more inner surfaces of the reaction cell of the sensor chip disposed towards one or more functional surfaces of the reaction cell. By mechanically coupling the piezo elements to the at least one functional surface or one or more inner surfaces of the reaction cell, the mass and/or change in mass of receptor protein complex immobilized at or released from the at least one functional surface may be reliably detected with high precision. As noted above, the one or more piezo elements may, for example, be arranged adjacent to a component carrying or supporting the at least one functional surface or the one or more inner surfaces of the reaction cell. For instance, if the at least one functional surface is provided or defined by an inner surface of the membrane, the one or more piezo elements may be arranged at or in contact with an opposite side of the membrane.

In an example, the receptor protein complexes may comprise at least one fluorescent label, wherein the sensing device may be configured to detect the one or more bioactive agents based on exciting the one or more fluorescent labels and based on detecting fluorescence light emitted by the one or more fluorescent labels. For instance, the receptor protein complexes may comprise at least one fluorescent label dissociating from a remaining part of the respective receptor protein complex upon binding to one or more of the bioactive agents, wherein the sensing device may be configured to detect the one or more bioactive agents based on exciting one or more fluorescent labels dissociated from one or more receptor protein complexes and based on detecting fluorescence light emitted by the one or more dissociated fluorescent labels. Accordingly, the sensing device may be configured to detect the state change induced by binding of the receptor protein complexes to one or more bioactive agents based on fluorescent excitation of free fluorescent or light absorbing labels within the reaction cell that are released from the receptor protein complexes upon binding.

The sensing device may include one or more light sources, for example laser diodes or light emitting diodes (LEDs), configured to excite the one or more fluorescent labels, wherein the sensing device may include one or more photodetectors configured to detect fluorescence light emitted by the one or more fluorescent labels.

Alternatively or additionally, the sensing device may include one or more light sources configured to illuminate at least a part of the reaction cell, wherein the sensing device may include one or more photodetectors configured to detect light scattered by one or more components of the receptor protein complexes within the reaction cell. Accordingly, the sensing device may be configured to detect the state change based on detecting scattered light and/or based on light scattering.

In an example, the one or more photodetectors may be arranged adjacent to or in proximity of a socket of the sensing device for at least partly receiving the sensor chip to detect electromagnetic radiation penetrating out of the reaction cell. For example, the one or more light sources may be arranged adjacent to or in proximity of the socket of the sensing device for at least partly receiving the sensor chip, such that electromagnetic radiation emitted by the one or more light sources is couplable into the reaction cell.

Alternatively or additionally, the one or more photodetectors may be arranged to oppose a detection window of the sensor chip. In other words, the one or more photodetectors may be arranged opposite to at least one detection window of the sensor chip, when the sensor chip is at least received by the sensing device.

In an example, the one or more light sources may be arranged to couple electromagnetic radiation or light through one or more optical connectors of the sensor chip into the reaction cell.

Alternatively or additionally, the sensing device may include one or more light sources arranged to couple electromagnetic radiation into one or more optical guides traversing at least a part of the reaction cell of the sensor chip, wherein the sensing device may include one or more photodetectors configured to detect electromagnetic radiation traversing the one or more optical guides. In an example, the one or more light sources may be arranged to align with an end of the one or more optical guides. Alternatively or additionally, the one or more photodetectors may be arranged to align with an end of the one or more optical guides. Such configuration may allow for a reliable optical measurement or detection of the state changes of the receptor protein complexes with accuracy, sensitivity and precision.

In an example, the processing circuitry may be configured to determine one or more optical properties of at least one of a functional surface and an inner surface of the reaction cell arranged adjacent the one or more optical guides. For instance, the processing circuitry may be configured to determine the one or more optical properties based on determining absorption of evanescence light penetrating through the one or more optical guides into the reaction cell. Also measuring optical properties of the functional surface or an inner surface of the reaction cell arranged adjacent the at least one optical guide can allow for a reliable optical measurement or detection of the state changes of the receptor protein complexes with high accuracy, sensitivity and precision.

In yet a further example, the sensing device may further include a housing configured to at least partly encompass the sensor chip, wherein the housing includes one or more holes for passing surrounding medium therethrough, such that at least a part of the sensor chip is contactable with the surrounding medium. Providing the one or more holes in the housing may ensure that the membrane of the sensor chip can be reliably contacted with the surrounding medium, thereby ensuring that bioactive agents can enter the reaction cell from the surrounding medium and be detected therein.

Optionally, the sensing device may further include one or more channels for passing surrounding medium through the one or more holes in the housing of the sensing device to said at least part of the sensor chip. In other words, the one or more holes arranged in the housing of the sensing device may be fluidly coupled via the one or more channels, such that surrounding medium can be conveyed through the holes and channels to the sensor chip or the at least part thereof.

Optionally, the sensing device may further include one or more ventilation devices arranged in the one or more channels and configured to convey or transport the surrounding medium through the one or more holes in the housing of the sensing device to said at least part of the sensor chip. For example, one or more micro-ventilators may be arranged in the one or more channels. Such configuration may prevent the surrounding medium from stagnating and may ensure that the sensor chip or the at least part thereof can be contacted with surrounding medium currently present in the surrounding of the sensing device.

In yet a further example, the sensing device may include one or more heating elements configured to heat at least a part of the sensor chip. The one or more heating elements may ensure that the sensor chip, the reaction cell or at least a part thereof can be kept at or near a predefined operational temperature of the sensor chip, for example at or near a body or room temperature. For instance, some mechanisms and detection principles applied may involve diffusion process, such as diffusion of bioactive agents into the reaction cell and diffusion of one or more components of the receptor protein complexes with the reaction cell. Such processes are in general temperature dependent and may require a minimum temperature to be maintained in order to get a detection signal within a reasonable time. Also the state changes of the receptor protein complexes may be temperature dependent and may be better observable at, near or above the predefined operational temperature of the sensor chip. Arranging heating elements at the sensing device may be of particular advantage in cold regions or during cold seasons, as proper functioning of the sensor chip and the sensing device can be ensured by heating the sensor chip.

For instance, the one or more heating elements may be arranged adjacent to a socket of the sensing device for at least partly receiving the sensor chip. Accordingly, the sensor chip or at least a part thereof can be efficiently heated by the one or more heating elements.

Optionally, the sensing device may further include one or more temperature sensors configured to determine a temperature of at least a part of the sensor chip. By means of the temperature sensors, a comprehensive temperature control can be implemented in the sensing device to ensure that the sensor chip, the reaction cell or at least a part thereof can be kept at or near the predefined operational temperature of the sensor chip.

In an example, the processing circuitry may be configured to control a temperature of at least a part of the sensor chip based on determining a temperature of said at least part of the sensor chip using the one or more temperature sensors. For instance, the processing circuitry may be configured to control the temperature of at least a part of the sensor chip, such that the reaction cell or at least a part thereof can be kept at or near the predefined operational temperature of the sensor chip. Optionally, the predefined operational temperature may be stored at a data storage of the sensing device and/or the sensor chip, which may accessed by the processing circuitry during operation to control the temperature.

In a further example, the sensing device further includes one or more electrical connectors for connecting to one or more electrical connectors of the sensor chip upon insertion of the sensor chip into the sensing device. Preferably, electrical connection between the sensor chip and the sensing device can be automatically established upon insertion of the sensor chip into the sensing device.

In an example, the processing circuitry may be couplable to one or more electrodes of the sensor chip, wherein the processing circuitry may be configured to determine a conductivity of a substrate or composition within the reaction cell. Accordingly, the processing circuitry may be configured to control the one or more electrodes, for example based on supplying voltage and/or current to the one or more electrodes, to perform a conductivity measurement within the reaction cell to detect the state change induced by binding of the receptor protein complexes to one or more bioactive agents.

Alternatively or additionally, the processing circuitry may be configured to determine one or more operational parameters indicative of a usage or remaining lifetime of the sensor chip. Alternatively or additionally, the processing circuitry may be configured to determine a usage or a remaining lifetime of the at least sensor chip based on determining one or more operational parameters of the sensor chip. As noted above, for example due to depletion of receptor protein complexes, a lifetime of the sensor chip may be limited. By determining the remaining lifetime or usage of the sensor chip, for example a user may be notified when the sensor chip should be replaced. Also, the sensor chip may be used multiple times and for example be re-inserted into the sensing device, while ensuring that the sensor chip is still operational or functional.

In an example, the processing circuitry may be configured to determine the usage or remaining lifetime of the at least one sensor chip based on retrieving historic data indicative of the usage or remaining lifetime of the sensor chip from a data storage of the sensor chip. Accordingly, historic data may be stored at the sensor chip and may be processed by the processing circuitry of the sensing device to determine the usage or remaining lifetime of the sensor chip. The historic data may, for example, include values for one or more operational parameters, for example accumulated over a previous or past time of operation of the sensor chip.

For example, the processing circuitry may be configured to determine the usage or remaining lifetime of the at least one sensor chip based on comparing the determined one or more operational parameters of the sensor chip and/or at least a part of historic data retrieved from a data storage of the sensor chip with one or more threshold values. The one or more threshold values may be stored at a data storage of the sensing device or may be retrieved by the processing circuitry from a data storage of the sensor chip.

Optionally, the processing circuitry may be configured to determine the usage or remaining lifetime of the at least one sensor chip based on determining a type of the sensor chip and/or a type of receptor protein complexes employed in the sensor chip. The type of sensor chip and/or receptor protein complexes employed therein may be determined based on reading a chip identifier of the sensor chip, based on retrieving corresponding data from a data storage of the sensor chip, and/or based on a user input, for example via a user interface of the sensing device.

Further, the processing circuitry may optionally be configured to store the one or more operational parameters in a data storage of the sensing device and/or a data storage of the sensor chip. Accordingly, the one or more operational parameters can be stored, for example as historic data, which allows for a determination of the usage and/or remaining lifetime of the sensor chip at a later time.

In an example, the processing circuitry may be configured to determine one or more operational parameters based on monitoring a temperature of at least a part of the sensor chip. In other words, the one or more operational parameters can include a temperature of at least a part of the sensor chip, for example a temperature over time. Since certain processes or reactions within the reaction cell, such as diffusion as well as depletion of receptor protein complexes, may be temperature dependent, the temperature of at least a part of the sensor chip over time may allow to accurately determine the usage and/or remaining lifetime of the sensor chip.

Alternatively or additionally, the processing circuitry may be configured to determine depletion of receptor protein complexes in the sensor chip over time. For instance, the processing circuitry may be configured to determine depletion of the receptor protein complex based on one or more operational parameters of the sensor chip and/or the sensing device over time, for example based on a temperature of at least a part of the sensor chip over time. Optionally, the processing circuitry may be configured to determine a remaining lifetime of the sensor chip based on the determined depletion of receptor protein complexes in the sensor chip over time.

In a further example, the sensing device may include a user interface, wherein the processing circuitry may be configured to provide a notification to a user via the user interface upon expiry of a lifetime of the sensor chip. Hence, the user may be notified upon expiry of the sensor chip lifetime, such that the sensor chip can be replaced by the user in time.

Optionally, the processing circuitry may be configured to provide information related to the determined one or more bioactive agents to a user via the user interface. For instance, information on an immediate or current quality of the surrounding medium, for example an air quality, and/or an immediate exposure to bioactive agents may be provided at the user interface. Also other information, such as an exposure in the past, or other information related to operation of the sensor chip and/or the sensing device, such as information about a usage and/or remaining lifetime of the sensor chip, may be provided at the user interface.

Further optionally, the processing circuitry may be configured to notify the user via the user interface upon determining a concentration of bioactive agents in the surrounding medium reaching or exceeding a predefined threshold. Accordingly, a warning can be provided to the user upon detecting a high concentration of bioactive agents.

Generally, the user interface may be any type of user interface. For instance, the user interface may include one or more of a vibrational element, a display, one or more LEDs, and a speaker.

In yet another example, the sensor chip may include a chip identifier for identifying a type of the sensor chip and/or a type of receptor protein complex contained in the sensor chip, wherein the processing circuitry may be configured to retrieve information or data indicative of a type of the sensor chip and/or a type of receptor protein complex contained in the sensor chip from the chip identifier. The sensing device may optionally include one or more of a barcode reader, a QR code reader, and an RFID scanner or reader for this purpose.

The sensing device may further include one or more energy storages for supplying electrical energy. For example, the sensing device may comprise one or more batteries, accumulators and/or capacitors for supplying electrical energy to the sensing device and/or the sensor chip. Such configuration may be of particular advantage when the sensing device is designed as portable or mobile device. Alternatively or additionally, however, the sensing device may be supplied with electrical power via a supply grid.

The sensing device may further include communication circuitry for communicatively coupling the sensing device with a user device. For example, the communication circuitry may include a wireless communication circuitry, preferably a Bluetooth transmitter, infrared transmitter or Wireless LAN transmitter. This may allow for a wireless data connection and communication between the sensing device and the user device. In particular, this may allow to retrieve data from the sensing device, for example data indicative of the exposure with bioactive agents and/or indicative of a quality of the surrounding medium, with the user device. The data may then be further processed and/or displayed at the user device and can be evaluated by the user.

In another example, the sensing device may further include one or more radiation sensor for measuring exposure of the sensing device to ionizing radiation. For instance, the one or more radiation sensors may include at least one of a reversibly photochromic layer sensitive to UV radiation and a radiochromic dye film sensitive to ionizing radiation. Using one or more radiation sensors may allow to determine the radiation exposure in parallel to or simultaneously with the determination of the bioactive agents. Hence, quality of the surrounding medium and/or an exposure of the user to various environmental parameters can be comprehensively assessed.

Alternatively or additionally, the sensing device may further include one or more of a humidity sensor, a Volatile Organic Carbon sensor, an Ozone sensor, a particulate matter sensor, a sensor for nitric acid, and a carbon monoxide sensor. Also using one or more of the aforementioned sensors may allow to comprehensively determine or assess an exposure of the user to other various environmental parameters in addition to the exposure with bioactive agents.

According to a further aspect of the present disclosure, there is provided a sensing system for detecting one or more bioactive agents in a surrounding medium. The sensing system comprises at least one sensor chip, as described hereinabove and hereinbelow, and a sensing device, as described hereinabove and hereinbelow. Any disclosure presented hereinabove and hereinbelow with reference to any of the sensing device and the sensor chip equally applies to the sensing system.

Further aspects of the present disclosure relate to a use of a sensing device, as described hereinabove and hereinbelow, and to a use of a sensing system, as described hereinabove and hereinbelow, for detecting one or more bioactive agents in a surrounding medium.

According to a further aspect of the present disclosure, there is provided a method of detecting one or more bioactive agents in a surrounding medium with or using one or more of a sensing system, a sensing device, and a sensor chip, as described hereinabove and hereinbelow. Alternatively or additionally, the method may relate to a method of operating one or more of the sensing system, the sensing device, and the sensor chip. The method comprises the following steps:
passing one or more bioactive agents through a membrane of the sensor chip into a reaction cell of the sensor chip, for example based on diffusion;
binding one or more bioactive agents to one or more receptor protein complexes, thereby inducing a state change of at least a part of the receptor protein complexes;
detecting, with the sensing device, the sensor chip and/or the sensing system, the induced state change of said at least a part of the receptor protein complexes; and
generating, with the sensing device, the sensor chip and/or the sensing system, a detection signal indicative of presence of the one or more bioactive agents in the surrounding medium based on the determined state change of said at least a part of the receptor protein complexes.

Again, it is emphasized that any disclosure presented hereinabove and hereinbelow with reference to any of the sensing device, the sensor chip, and the sensing system equally applies to the method of detecting one or more bioactive agents.

In an example, passing one or more bioactive agents through the membrane of the sensor chip includes contacting a membrane of the sensor chip with the surrounding medium.

Optionally, the method may further comprise activating the sensor chip based on supplying de-ionized water from a reservoir of the sensor chip to the reaction cell of the sensor chip.

For instance, activating the sensor chip may include unblocking fluid communication between the reaction cell and the at least one reservoir using or based on actuating at least one blocking element between the reservoir and the reaction cell of the sensor chip. Optionally, the at least one blocking element may include a movable pin arranged between the reservoir and the reaction cell, wherein activating the sensor chip may include displacing the movable pin.

Alternatively or additionally, the at least one blocking element may include a water-impermeable membrane arranged between the reservoir and the reaction cell, wherein activating the sensor chip may include disrupting at least a part of the water-impermeable membrane.

Alternatively or additionally, activating the sensor chip may include removing a sealing cover from an outer surface of the membrane. By removing the sealing cover, contact between the membrane and the surrounding medium may be established, such that bioactive agents to be detected can enter the reaction cell through the membrane.

In yet another example, detecting the induced state change may include determining one or more of a number, a mass, a density, and a mass density of receptor protein complexes dissolved in a substrate of the reaction cell of the at least one sensor chip. Alternatively or additionally, detecting the induced state change may include determining one or more of a number, a mass, a density, and a mass density of receptor protein complexes immobilized at or liberated from at least one functional surface of the reaction cell.

Alternatively or additionally, detecting the induced state may include determining one or more of a number, a mass, a density, and a mass density of receptor protein complexes immobilized at or liberated from at least one inner surface of the reaction cell disposed towards at least one functional surface of the reaction cell. Alternatively or additionally, detecting the induced state change may include exciting one or more fluorescent labels of the receptor protein complexes and detecting fluorescence light emitted by the one or more fluorescent labels. Alternatively or additionally, detecting the induced state change may include detecting light scattered by one or more components of the receptor protein complexes within the reaction cell. Alternatively or additionally, detecting the induced state change may include determining one or more optical properties of at least one of a functional surface and an inner surface of the reaction cell. Alternatively or additionally, detecting the induced state change may include determining absorption of evanescence light penetrating through one or more optical guides of the sensor chip into the reaction cell.

In the following, various aspects, examples and exemplary embodiments of the present disclosure are summarized, which is to be construed non-limiting and serves illustrative purposes only.

The sensor chip, the sensing device and the sensing system described herein may employ receptor protein complexes, such as for example biological receptor proteins, as a detection element or chemical recognition element. The receptor protein complexes may be present in an aqueous environment, for example in the reaction cell of the sensor chip, and may interact with the surrounding medium through a porous membrane, for example of low diffusion resistance. The receptor protein complexes may, for example, be or comprise xenosensor proteins or hormone receptors such as AHR, CAR, PXR and ER. This list of candidate receptor protein complexes is not exhaustive and any other protein displaying a binding specificity towards one or several ligand molecules may serve as a chemical recognition element.

The present disclosure may not be limited to personalized air quality monitoring, but may also be applied for, for instance, detecting pollutants in water or soil extracts. In addition, proteins active in homeostatic biochemical processes, for instance metabolism, may be used, in which case the sensing system may be used for diagnostic applications.

Moreover, the gene sequence usable for cloning the receptor protein complexes can be of human, animal, plant, fungal bacterial or archeal origin. For instance depending on the working principle of the sensor chip on the molecular level, they can be used in non-recombinant form or in form of recombinant proteins. Furthermore, combinations of non-recombinant and recombinant proteins and combinations of different proteins of the same or different species origin may be used as at least a part of the receptor protein complexes. Optionally, receptor protein complexes may be chemically labelled after their isolation, for instance with fluorophores. As used herein, candidate proteins are collectively termed receptor protein complexes, irrespective of their biological origin, on whether they are recombinant or not and on whether they are labelled or not. In addition, proteins that in the living cell may interact in some way with the receptor protein complexes, but may themselves not be receptor proteins, may be included. Such additional proteins may be of any origin, recombinant or non-recombinant, labelled and unlabelled.

As described hereinabove, the sensing system may be stationary or portable devices, for example for environmental monitoring. The sensing system may comprise one or more of the features described in the following. The sensing system may comprise one or more exchangeable and/or disposable sensor chips, which can be mounted on the sensing device, for instance by a click mechanism or a magnetic connection. The sensor chip may contain the receptor proteins in a suitable liquid, semisolid and/or gelatinous matrix. The sensor chip may provide appropriate electrical and/or optical contacts for connecting it to the sensing device. The sensor chip may provide a gas permeable surface at which mass transfer from the surrounding medium or atmosphere into the reaction cell occurs. Optionally, the chip may contain parts or the majority of the transducer and/or the processing circuitry that may convert the event of state change or ligand binding to a receptor protein complex into an electronically processable variable or signal. The sensing device may further comprise a socket into which sensor chip can be inserted. The socket may provide appropriate optical and/or electrical and/or mass flow connections required for operating and maintaining the sensor chip and may allow the gas permeable surface of the sensor chip to be in direct and optionally continuous contact with the surrounding medium atmosphere. The sensing device may optionally comprise one or more of an outer housing, an on/off button located on the outer housing, a connection for plugging a power source for battery charging, embedded in the outer housing, perforations in the outer housing, allowing surrounding air to enter the outer housing, and a small screen or arrays of LEDs embedded in the outer housing allowing the device to give a color-coded light signal describing the level of air pollution in the surroundings and the device state, such as battery status. The sensing device may optionally comprise one or more of a speaker allowing the device to give acoustic signals if a high air pollution in the surroundings is detected, a means for fixing the device on, for instance, wears, bikes or straps of backpacks, a reversibly photochromic coating or layer sensitive to UV radiation, and a radiochromic dye film sensitive to ionizing radiation. The sensing device may optionally comprise one or more of a source of a physical signal, such as a light source, voltage source, and a current source, which upon the event of binding of a bioactive agent to a receptor protein complex experiences a measurable change in its properties, such as in intensity, amplitude, wavelength, and/or frequency. The majority or the entirety of the transducer and/or the processing circuitry that detects the measurable change in this physical signal and converts it into an electronically processable variable or signal may be comprised in the sensing device. The sensing device may optionally further comprise one or more of a micro-fan or ventilation device that may actively sample surrounding medium and supplies it to the sensor chip(s). Also, the sensing device may comprise means for controlling the sampling flow rates generated by the micro-fan may be included in the sensing device. Further optionally, a Bluetooth transmitter and/or receiver or other means of communication with external devices, such as a user device. Moreover, the sensing device may comprise one or more of a rechargeable battery pack, a microprocessor, microelectronics and micro-optics for operating sensor chips, for monitoring sensor chip status and/or for reading the response of the chips to pollution of the surrounding medium. Moreover, the sensing device may comprise one or more of a temperature sensor, a heating unit or element, a data storage, one or more sensors for sensitive to humidity, pressure, VOC, ozone, nitric oxides, carbon monoxide, particulate matter, or the like, and optional accessory components. The sensing device may further comprise one or more of a GPS tracker, a clock, and means for sharing collected information on quality of the surrounding medium, on time and/or on location in a larger, for example cloud-based network. Further, a software or application may be provided at a user device to control the sensing system, the sensing device and/or the sensor chip and obtain sensor readings, warnings, and information on current and predicted quality of the environment. The software or application may be configured to access the shared data and use it for, for instance, estimating or predicting a quality of the surrounding medium at a given place and time with the objective of, for instance, route planning or developing exposure avoidance strategies. The software or application may be configured, for instance, monitor cumulative personal exposure, to compile reports on the success of exposure avoidance strategies or to correlate exposure with health state, for example monitored by any compatible health monitoring device.

In the following, the technical concept envisaged by the present disclosure is summarized for illustrative purposes, which is to be construed non-limiting. Molecules, compounds and/or agents present in the medium surrounding the sensing device can diffuse into the reaction cell of the sensor chip. If such molecules are ligands of the receptor proteins or receptor protein complexes contained in the reaction cell, they can bind the receptor protein complexes and induce changes to the state of the receptor protein complexes that can be measured. A possible technical solution can comprises a gas-permeable membrane for the sensor chip, such as porous carbon papers used in fuel cells or microperforated fluoropolymers, both of which are of low diffusion resistance and largely impermeable to aqueous liquids. The gas-permeable membrane may separate the surrounding medium from the reaction cell, which for example can contain a liquid, semisolid and/or gelatinous substrate containing the receptor protein complexes. For instance, the receptor protein complexes may comprise the ligand binding domain of a xenosensor protein, for instance the human AHR.

Alternatively or additionally, at least some of the receptor protein complexes may comprise at least one binding domain of an aryl hydrocarbon receptor or aryl hydrocarbon receptor protein. For instance, at least some of the receptor protein complexes may comprise at least the PAS (Per-ARNT-Sim) domain or the entire aryl hydrocarbon receptor protein, which may allow for detection of materials, compounds, molecules and/or agents, for example halogenated aromatic hydrocarbons, such as polychlorinated dibenzodioxins, dibenzofurans and biphenyls, and/or polycyclic aromatic hydrocarbons, such as 3-ethylcholanthrene, benzo[a]pyrene, benzanthracenes and benzoflavones.

The receptor protein may further be immobilized at a specific location within the reaction cell of the sensor chip by covalently linking low affinity ligands of the receptor protein complexes to a functional surface of the reaction cell, such as an inner surface of the reaction cell or the membrane. Binding of the ligands by the receptor protein complexes may immobilize the receptor protein complexes. Further, medium or high affinity ligands, such as the bioactive agents, may diffuse into the reaction cell and may competitively bind to the receptor protein complexes and liberate them from the site of immobilization.

The event of liberation can be detected by various means. For instance, the mass bound to the functional surface or another inner surface of the reaction cell can be monitored by piezo elements, such quartz crystal microbalances. Alternatively or additionally, optical properties of the functional surface or another inner surface of the reaction cell can be monitored by measuring interaction of molecules in close vicinity of the surface of a waveguide or optical guide with an evanescent light field penetrating the surface. Alternatively or additionally, the presence of liberated receptor protein complexes rather than the liberation itself can be detected. For example, the receptor protein complexes may be linked with a label that is detectable when the receptor protein complex is diffusing freely in the reaction cell. Labelling may, for instance be achieved in the form of fusion proteins and the green fluorescent protein (GFP), in which case the detection may be based on fluorescent excitation of GFP and detection of the emitted fluorescent light by one or more photodetectors of the sensing device. Human xenosensor proteins as well as various recombinant versions thereof, including fusion proteins containing the green fluorescent protein GFP, can be cloned and used in a molecular binding assay. A number of potentially suitable low affinity ligands, such as AHR ligands, may be used. The sensitivity of the sensing device may depend on the sensitivity of nowadays available detectors, such as piezo elements or photodetectors, and the processing circuitry. Such elements are of sufficient sensitivity for detecting the liberation of receptor protein complexes. For detection by piezo elements, for instance, the receptor protein complexes can be linked to high mass moieties such as nanoparticles or gold nanoparticles, which can result in detection limits in the 50 femtomolar concentration range. For optical detection, the signal per receptor protein complex can be increased by orders of magnitude by using fluorescent mechanisms, which allow for repeated excitation and emission of light. Several detailed examples of working principles of on the molecular level are hereinabove and hereinbelow. It is noted that the chemical structure of the bioactive molecules or agents that bind to the receptor protein complexes and trigger the sensor chip or sensing device to report a detection signal may not be known. The sensor chip and sensing device according to the present disclosure are therefore superior to sensors that selectively detect a chemical compound, which may be known or assumed to have adverse effects towards human health. On the other hand, the bioactive molecules or agents should preferably bind the receptor protein complexes with sufficient affinity to induce the measurable change in the state of the protein complexes. It can therefore be assumed that it would do so in the living organism too, which would either trigger cellular defense mechanisms (if binding to a xenosensor protein) or it would activate cellular responses in the absence of the activity of the endogenous signalling (if binding to a hormone receptor). The sensor chip and sensing device are therefore also superior to sensors that non-selectively respond to a broad range of chemical compounds based on certain structural properties which may not be related to the compounds' bioactivity.

It is worth noting that while for conventional sensors, the unit of measure may be the concentration of a given compound in the surrounding medium or the mass concentration of constituents of specified physical or chemical properties, the concept of mass per volume or parts per volume may not suitably apply for the sensor chip and sensing device described herein. A readout or detection signal intensity generated by sensing device may be the result of the concentration of bioactive agents in the surrounding medium and their affinity to the receptor protein complexes. The unit of measure of the sensor chip and sensing device described herein may therefore best be described as the receptor protein activation potential of the surrounding medium, hence the bioactivity of the surrounding medium, for example with respect to the chosen type of receptor protein complex.

In the following, the details of the technical concept envisaged by the present disclosure are summarized for illustrative purposes, which is to be construed non-limiting. The sensor chip may basically have any shape and size. In particular for portable sensing devices, a footprint of the sensor chip may be minimized. By way of example, the sensor chip may be about 1-10 cm in length, 1-10 cm in width and 0.5-2 cm in thickness, potentially depending on the design of the sensing device. The sensor chip comprises a reaction cell, a compartment containing a substrate, such as an aqueous, isotonic, buffered liquid, semisolid and/or gelatinous matrix, in which the receptor protein complexes and potential additional proteins may maintain their native state. The substrate may be at one or several sides of the reaction cell separated from the surroundings by a membrane permeable to gases, but not to water. The thickness and structure of the membrane material may be chosen to minimize the resistance to diffusion. Specifically, the membrane be preferably of low thickness and/or high porosity. Optionally, the pores of the membrane may exclude water, this is, they may be filled with gas. The gas-permeable membrane may be protected from physical damage from the outside, for instance by a rigid open grid, which may be part of the housing of the chip and may allow surrounding gas from the medium to freely access the membrane. The grid may be covered by and adhesive film or an air-tight cover when the sensor chip is in its unused state. This can prevents molecules or agents from the surrounding to enter the cell when it is not in operation. The cell may be re-sealable, which can allow to extend the life span or lifetime of the reaction cell.

Furthermore, as water may evaporate from the reaction cell by passing through the gas-permeable membrane, the sensor chip may provide a small reservoir containing de-ionized water. The reservoir may be connected to the reaction cell, wherein diffusion of salts from the reaction cell may be hindered by the presence of a semi-permeable membrane at the connection. In an example, the reservoir may be a small, flexible blister that does not pose any significant resistance to deflation if water is leaving towards the reaction cell. The reservoir may be surrounded by a rigid housing, which may comprise holes through which surrounding air or medium can enter the housing when water is pulled from the flexible blister towards the reaction cell by the higher osmolarity in the reaction cell. Alternatively or additionally, the flexibility of the volume of the water reservoir may be achieved by means of a movable piston.

In an example, specifically if the receptor protein complexes and potential additional proteins maintain their functionality when lyophilized and reconstituted, the same water reservoir or another reservoir may serve for activation of the sensor chip. Activation may be achieved by destroying a water-impermeable barrier or membrane between the reservoir and the reaction cell, upon which water will enter the reaction cell and reconstitute the receptor protein complexes. This process may be driven by the higher osmolarity of the reaction cell and will continue until the osmotic pressure in the reaction cell is counter-balanced by its housing and optionally the rigid grid covering the gas-permeable membrane.

Furthermore, the sensor chip may provide electrical and/or optical contacts or connection through which it can be connected to the sensing device when mounted.

Receptor protein complexes may be cloned, expressed and isolated and/or purified using standard molecular biological methods. Depending on the the molecular working principle of the sensor chip, accessory proteins that in the living cell are i) bound to the receptor protein complexes and dissociate from it upon ligand binding or ii) bind to the receptor protein complexes upon ligand binding or iii) chemically modify the receptor protein upon ligand binding or iv) are chemically modified by the receptor protein complexes upon ligand binding may be cloned and isolated and included in the sensor chip in addition.

In the functional device, the receptor protein complexes may be present in the reaction cell of the sensor chip. Depending on the molecular working principle of the sensor chip, they may be immobilized, that is, confined to a region or surface inside the reaction cell or dissolved in the substrate contained therein. Further, depending on the working principle of the sensor chip on the molecular level, the dissolved or immobilized receptor protein complexes may be in their monomeric form (not in complex with other proteins), as homodimers (two receptor proteins bound together) or in form of multiprotein complexes.

In the example where receptor protein complexes are immobilized to a region or surfaces of the reaction cell, the receptor proteins may preferably be immobilized in closest possible proximity to where constituents of the surrounding medium enter the cell. For example, this can be the gas-permeable membrane itself, specifically the side of the membrane facing the inside of the reaction cell. Such an embodiment may minimize the delay between a constituent of the surrounding medium, such as a bioactive agent, entering the reaction cell and its binding to a receptor protein complex and can simultaneously maximize the likelihood of the binding, as the point of entry is tightly packed with the receptor protein complexes.

Immobilization of receptor protein complexes can be achieved by covalently binding the receptor protein complexes or one member of homodimers or one or several members of multiprotein complexes to the membrane. The covalent link thereby may not alter the structure of ligand binding domains and/or sites of protein-protein interaction and may not sterically affect protein or ligand binding. Alternatively or additionally, it can be achieved by immobilizing a low affinity ligand of the used receptor protein complexes (if only one receptor protein type is present), or a mixture of low affinity ligands (if several different receptor protein types are present). Alternatively or additionally, it can be achieved by covalently linking molecules that bind specific regions or domains of the receptor protein complexes or one or several of the additional proteins which are present in the reaction cell and bound to the receptor protein complexes. The targeted regions or domains of the binding proteins may thereby not be identical to the ligand binding site or the site of interaction between different proteins present in the reaction cell. Examples include the immobilization of monoclonal antibodies specific to a certain epitope of the protein to be immobilized, or the immobilization of nitrilotriacetic acid-coupled nickel²⁺ ions, if the protein complex to be immobilized contains the histidine-tag able to chelate nickel ions.

Several means for immobilization of proteins with the reaction cell can be implemented. These may serve the same purpose, such as immobilization of the protein complexes before ligand binding, or different purposes, such as binding of protein complexes that have bound a ligand or bioactive agent. Also, several types of receptor protein complexes may be present in the same sensor chip, which can allow broadening the chemical specificity of the sensor. Different labels may be linked to the receptor protein complexes and/or the additional proteins that interact with receptor protein complexes, which allows determining which of the several used receptor protein complexes did bind a ligand or corresponding bioactive agent present in the surroundings. Alternatively or additionally, various chips containing different receptor protein complexes, hence providing different specificity, may be available, optionally all matching the socket present on the sensing device.

Once activated, the sensor chip may have a limited lifespan or lifetime because of receptor protein complex depletion, this is the amount of receptor protein available for environmental monitoring may decrease with time. This decrease can be attributed to two mechanisms: i) at some point, all available receptor proteins will have bound a molecule that entered the reaction cell from the surroundings and ii) the receptor proteins have a defined half-life even in absence of binding molecules or agents and in the optimized physicochemical conditions in the substrate of the sensor chip. Receptor protein complex depletion by the former mechanism may be a function of the cumulative detected pollution and can be calculated based on the known amount of receptor protein complexes initially present in the sensor chip. Receptor protein complex depletion by the latter mechanism may be a function the receptor protein stability under the physicochemical conditions within the reaction cell. It may be defined by parameters specific to the sensor chip, such as the employed receptor protein complex and the substrate composition, and by parameters specific to the use of the sensing device, such as the temperatures the sensor chip was exposed to during its lifetime. Receptor protein complex depletion can be determined continuously or at defined time intervals by the sensing device. In embodiments employing immobilized receptor protein complexes, for instance, this may be achieved by measuring properties such as the impedance of the absorbance of the layer of immobilized receptor protein complexes. Alternatively or additionally, parameters specific to the sensor chip may be determined as part of the development or production. Further, parameters related to the use of the sensing device or sensor chip may be measured throughout the life span of the chip.

The sensor chips may for example carry a chip identifier which can allow the software application used for operating the sensing device, for instance installed on a user device or on the sensing device, to read the history of use of the chip. The chip identifier may preferably be detected by the sensing device automatically upon chip insertion. Alternatively or additionally, the chips may carry physical identifiers, such as a chip ID number, which, upon inserting the chip in the sensing device, may be entered by the user. From the history of use, the sensor type and cumulative detected pollution or exposure, the software can estimate the receptor protein depletion of the sensor chip and notify the user when the chip should be exchanged.

Due to the limited life span of a sensor chip and for reasons of sustainability and product costs, the sensors chips may preferably be made of the smallest possible amount of, preferably recycled and recyclable, material. The chips preferably only contain electrical and/or optical leads or connectors, but no further electronic sensor components involved in signal generation, capturing or processing, such as diodes, photodetectors, or processors. Alternatively, however, one or more of such components may be included in the sensor chip.

In the following, a sensor response time is exemplary estimated. The basic working principle may include transport of a bioactive agent from the surrounding medium to the boundary layer at the gas-permeable membrane, transport across the boundary layer to the gas permeable membrane, diffusion of the bioactive agent through the gas permeable membrane, diffusion of the bioactive agent to the receptor protein complexes, binding of the bioactive agent to the receptor protein complexes, optionally diffusion of the receptor protein complex to the site of detection, and finally detection of the state change induced upon binding.

The transport of a bioactive agent from the surrounding medium to the boundary layer at the gas-permeable membrane may be driven by convection, that is, surrounding medium may be brought actively or passively to the boundary layer at the gas-permeable membrane. With the specified device dimensions and sampling rates of about 0.5 to 10 mL/seconds, which may depend on the size of the device, both passive and active mass transport can be expected to cause a delay of approximately one to five seconds relative to the occurrence of the bioactive agent in the surroundings of the sensor chip.

The transport across the boundary layer to the gas permeable membrane and the transport across this layer may be dependent on Brownian motion and on the diffusion coefficient D of the molecule or bioactive agent under consideration according to the equation D=msd(x)/2Δt, where msd(x) is the mean square displacement in x-direction (e.g. towards the gas permeable membrane) and Δt is the time. Diffusion coefficients of for relevant organic molecules and bioactive agents may be in the range of 0.05 cm2/sec (in air, at ambient conditions). Assuming a thickness of the open grid of 2 mm constituting the boundary layer, the transfer through the combs of the grid to the gas permeable membrane can be expected to occur during approximately 2 seconds.

Further, the transfer across the gas permeable membrane may follows the same physical principles, but depending on the pore size of the membrane, the diffusion coefficient D is to be replaced by the Knudsen diffusivity, i.e. the effective diffusivity is decreased as collisions of the molecule with the pore wall become more likely than collisions between the molecules in the fluid. In addition, depending on the membrane type, the pores of the membrane may be filled with liquid or gas and the according diffusion coefficient to be taken (Diffusion coefficients in water are by orders of magnitude lower). Assuming pores sizes in the membrane of about 0.2 μm, which is in the range of roughly half the mean free path of a molecule in air at 25° C., and air-filled pores and, the Knudsen diffusion coefficients are in the range of about 0.025 cm²/sec. A membrane of 200 μm thickness, thus, may cause a further delay of roughly 0.2 seconds.

The dynamics of the diffusion of the bioactive agents to receptor protein complexes may cause a further delay. It may be negligible in embodiments where the receptor protein complexes are immobilized at the gas permeable membrane. For embodiments in which the receptor protein complexes are dissolved in the reaction chamber, it can be estimated as described above, using diffusion coefficients in water and the average distance between the gas permeable membrane and the location of the dissolved receptor protein complexes. Diffusion coefficients of relevant organic molecules in water may be in the range of 10-5 cm²/sec, the average distance between a dissolved receptor protein complex and the gas permeable membrane can be estimated to be half the reaction cell diameter. The dimensions of the reaction cell can therefore be in the range of 0.1 mm or lower. In case of larger dimensions (an average distance of 1 mm, for instance, causes a delay of several thousand seconds), the substrate present within the reaction cell can be mixed actively, for instance by generating convective flow within the reaction cell or by a non-uniform supply of heat or by introducing a microfluidic circulation.

The actual binding between the bioactive agent and the receptor protein complex may be a fast process and can be neglected for the estimation of the response time. Further, for the diffusion of the receptor protein complex to the site of detection, the same considerations as described above regarding diffusion of the bioactive agent to the receptor protein complex apply, but diffusion coefficients of protein complexes may commonly be in the range of 10-6 cm²/sec, which is about tenfold smaller than diffusion coefficients of organic molecules or bioactive agents. Further, the detection commonly happens immediately and does not contribute to the delay between occurrence of a bioactive agent and the generation of a sensor signal.

Below, there is provided a non-exhaustive list of non-limiting examples. Any one or more of the features of these examples may be combined with any one or more features of another example, embodiment, or aspect described herein.

Example 1: A sensor chip operatively couplable to a sensing device for detecting one or more bioactive agents in a surrounding medium, the sensor chip comprising:
a reaction cell containing a plurality of receptor protein complexes; and
a membrane separating the reaction cell from the surrounding medium and being permeable for the one or more bioactive agents;
wherein the receptor protein complexes are configured to bind to the one or more bioactive agents in the reaction cell, such that a detectable state change of at least a part of the receptor protein complexes is induced.

Example 2: The sensor chip according to example 1, wherein the detectable state change of said at least part of the receptor protein complexes is indicative of a presence of the one or more bioactive agents in the surrounding medium.

Example 3: The sensor chip according to any one of the preceding examples, wherein the state change of the at least part of receptor protein complexes is associated with one or more of a change in a conformational state of the at least part of receptor protein complexes, a change in a localization of the at least part of receptor protein complexes within the reaction cell, a change in a position of the at least part of receptor protein complexes within the reaction cell, a change in a composition of the at least part of the receptor protein complexes, a change in a mass of the at least a part of the receptor protein complexes, a change in a mass of at least a part of the reaction cell, a change in a physical property of the at least a part of the receptor protein complexes, a change in a physical property of at least a part of the reaction cell, a change in an optical property of at least a part of the reaction cell, a change in a chemical property of the at least a part of the receptor protein complexes, a change in a chemical property of a substrate contained in the reaction cell, a change in a conductivity of a substrate contained in the reaction cell, and a change in a concentration of free fluorescent or light absorbing molecules within at least a part of the reaction cell.

Example 4: The sensor chip according to any one of the preceding examples, wherein the receptor protein complexes are configured to change a position within the reaction cell upon binding to the one or more bioactive agents.

Example 5: The sensor chip according to any one of the preceding examples, wherein the receptor protein complexes are configured to dissociate into one or more components upon binding to the one or more bioactive agents.

Example 6: The sensor chip according to any one of the preceding examples, wherein at least a part of the receptor protein complexes is confined to or immobilized at at least one functional surface of the reaction cell; and wherein the receptor protein complexes are configured to dissociate from the at least one functional surface upon binding to the one or more bioactive agents.

Example 7: The sensor chip according to any one of the preceding examples, wherein at least a part of the receptor protein complexes is covalently linked to at least one functional surface of the reaction cell.

Example 8: The sensor chip according to any one of the preceding examples, wherein at least a part of the receptor protein complexes is confined to or immobilized at at least one functional surface of the reaction cell by a low affinity ligand covalently linked to the at least one functional surface of the reaction cell.

Example 9: The sensor chip according to any one of examples 6 to 8, wherein the receptor protein complexes are electrostatically confined to the at least one functional surface of the reaction cell.

Example 10: The sensor chip according to any one of examples 6 to 9, wherein the at least one functional surface of the reaction cell is defined by an inner surface of the membrane facing the reaction cell.

Example 11: The sensor chip according to any one of examples 6 to 10, wherein the at least one functional surface includes an inner surface of the reaction cell disposed towards the membrane.

Example 12: The sensor chip according to any one of examples 6 to 11, wherein the at least one functional surface includes an inner surface of the reaction cell arranged opposite to the membrane.

Example 13: The sensor chip according to any one of the preceding examples, wherein the surrounding medium includes environmental air.

Example 14: The sensor chip according to any one of the preceding examples, wherein the surrounding medium includes water.

Example 15: The sensor chip according to any one of the preceding examples, wherein the membrane includes an outer surface configured to contact the surrounding medium; and wherein the membrane includes an inner surface facing the reaction cell.

Example 16: The sensor chip according to any one of the preceding examples, wherein the one or more bioactive agents includes an exogeneous agent.

Example 17: The sensor chip according to any one of the preceding examples, wherein the reaction cell contains one or more of a liquid, gelatinous, and semisolid substrate.

Example 18: The sensor chip according to example 17, wherein the substrate is composed such that the receptor protein complexes maintain a native state.

Example 19: The sensor chip according to any one of examples 17 and 18, wherein the substrate comprises at least one of an aqueous solution, an isotonic solution, and a buffered solution.

Example 20: The sensor chip according to any one of examples 17 to 19, wherein the substrate comprises a stabilizing protein for stabilizing the receptor protein complexes.

Example 21: The sensor chip according to any one of examples 17 to 20, wherein the substrate comprises a surface-active molecule or molecule complex.

Example 21: The sensor chip according to any one of examples 17 to 21, wherein the substrate comprises at least one of tween or triton X-100.

Example 23: The sensor chip according to any one of the preceding examples, wherein the membrane is permeable for gases; and/or wherein the membrane is impermeable for water or aqueous liquid.

Example 24: The sensor chip according to any one of the preceding examples, wherein the membrane includes a plurality of pores, preferably filled with gas to exclude water.

Example 25: The sensor chip according to any one of the preceding examples, wherein the membrane includes at least one of porous carbon paper material and perforated fluoropolymer.

Example 26: The sensor chip according to any one of the preceding examples, further comprising:
a grid covering at least a part of an outer surface of the membrane.

Example 27: The sensor chip according to any one of the preceding examples, further comprising:
a sealing cover covering at least a part of an outer surface of the membrane and configured to prevent contact of the membrane with the surrounding medium.

Example 28: The sensor chip according to example 26, wherein the sealing cover includes an adhesive film.

Example 29: The sensor chip according to any one of the preceding examples, wherein the reaction cell is sealable and/or re-sealable by covering at least a part of an outer surface of the membrane with a sealing cover.

Example 30: The sensor chip according to any one of 27 to 29, wherein the sealing cover is air-tight.

Example 31: The sensor chip according to any one of the preceding examples, wherein the sensor chip is configured in shape and size to be at least partly inserted into the sensing device.

Example 32: The sensor chip according to any one of the preceding examples, wherein the sensor chip is configured in shape and size to be at least partly inserted into a socket of the sensing device.

Example 33: The sensor chip according to any one of the preceding examples, wherein the sensor chip comprises at least one surface feature at an outer surface of a housing of the sensor chip, wherein the at least one surface feature of the sensor chip is formed complementary to at least one surface feature of the socket to ensure correct positioning of the sensor chip in the socket.

Example 34: The sensor chip according to example 33, wherein the at least one surface feature of the sensor chip is configured to engage with the at least one surface feature of the socket to fix the sensor chip in the socket.

Example 35: The sensor chip according to any one of the preceding examples, wherein the sensor chip includes one or more magnets for magnetically coupling the sensor chip to the sensing device.

Example 36: The sensor chip according to any one of the preceding examples, wherein the state change of the at least part of the receptor protein complexes is detectable based on an optical measurement, based on detecting fluorescent light, based on fluorescent excitation of one or more components of the receptor protein complexes, based on light scattering, based on determining a conductivity of a substrate contained in the reaction cell, based on an electrochemical process occurring in the reaction cell, based on determining one or more optical properties of at least one functional surface of the reaction cell, based on absorption of electromagnetic radiation, based on determining a mass of at least one functional surface of the reaction cell, based on determining a mass of receptor protein complexes confined to or immobilized at at least one functional surface of the reaction cell, and based on surface plasmon resonance at at least one functional surface of the reaction cell.

Example 37: The sensor chip according to any one of the preceding examples, further including:
one or more connectors for operatively coupling the sensor chip to the sensing device.

Example 38: The sensor chip according to example 36, wherein the sensor chip is connectable via the one or more connectors to the sensing device, such that the state change of the at least part of the receptor protein complexes upon binding to the one or more bioactive agents is detectable by the sensing device.

Example 39: The sensor chip according to any one of examples 37 to 38, wherein the one or more connectors include at least one optical connector configured to couple electromagnetic radiation from the sensing device into the reaction cell for optically detecting the state change of the at least part of the receptor protein complexes upon binding to the one or more bioactive agents.

Example 40: The sensor chip according to example 38, wherein the one or more connectors include at least one further optical connector for coupling electromagnetic radiation out of the reaction cell.

Example 41: The sensor chip according to any one of examples 38 to 40, wherein the at least one optical connector includes at least one aperture arranged in a housing of the sensor chip.

Example 42: The sensor chip according to example 41, wherein the at least one aperture is sealed with a layer of material translucent to electromagnetic radiation of a predefined wavelength.

Example 43: The sensor chip according to any one of examples 41 to 42, wherein the at least one aperture is oriented parallel to a longitudinal axis of the reaction cell.

Example 44: The sensor chip according to any one of the preceding examples, further including:
at least one optical guide for guiding electromagnetic radiation through the reaction cell for optically detecting the state change of the at least part of the receptor protein complexes upon binding to the one or more bioactive agents.

Example 45: The sensor chip according to example 44, wherein the at least one optical guide is aligned with and optically coupled to at least one optical connector of the sensor chip.

Example 46: The sensor chip according to any one of examples 44 to 45, wherein the at least one optical guide extends parallel to a longitudinal axis of the reaction cell through the reaction cell.

Example 47: The sensor chip according to any one of examples 44 to 46, wherein the sensor chip comprises at least two optical guides and at least one reflexive element arranged at an end of the at least two optical guides, the at least one reflexive element optically coupling the at least two optical guides.

Example 48: The sensor chip according to example 47, wherein each of the at least two optical guides is optically coupled with at least one optical connector of the sensor deceive arranged at an end of the respective optical guide opposite to the at least one reflexive element.

Example 49: The sensor chip according to any one of examples 37 to 48, wherein the one or more connectors include at least one electrical connector for electrically coupling the sensor chip to the sensing device.

Example 50: The sensor chip according to any one of the preceding examples, further including:
one or more electrodes at least partly arranged within the reaction cell and configured to determine a conductivity of a substrate or composition within the reaction cell.

Example 51: The sensor chip according to any one of the preceding examples, further including:
at least one detection window translucent for electromagnetic radiation emitted and/or scattered by at least one or more components of the receptor protein complexes.

Example 52: The sensor chip according to example 51, wherein the at least one detection window is translucent for fluorescent light emitted by the at least one or more components of the receptor protein complexes; and/or wherein the at least one detection window is opaque for fluorescence excitation light.

Example 53: The sensor chip according to any one of examples 51 to 52, wherein the at least one detection window is arranged opposite to and/or disposed towards the membrane.

Example 54: The sensor chip according to any one of examples 51 to 53, wherein an inner surface of the at least one detection window facing the reaction cell is at least partly coated with molecular trapping complexes configured to bind to at least a component of the receptor protein complexes, such that receptor protein complexes bound to the one or more bioactive agents are trapped at the inner surface.

Example 55: The sensor chip according to example 54, wherein the molecular trapping complexes comprise streptavidin.

Example 56: The sensor chip according to any one of examples 51 to 55, wherein an inner surface of the at least one detection window facing the reaction cell is at least partly coated with chelated nickel ions.

Example 57: The sensor chip according to any one of examples 51 to 56, wherein an inner surface of the at least one detection window facing the reaction cell is at least partly coated with quenching molecules for quenching fluorescent light emitted by at least one component of the receptor protein complexes.

Example 58: The sensor chip according to any one of the preceding examples, wherein an inner surface of the membrane facing the reaction cell is at least partly coated with quenching molecules for quenching fluorescent light emitted by at least a component of the receptor protein complexes.

Example 59: The sensor chip according to any one of the preceding examples, wherein the sensor chip and/or the reaction cell has an elongated shape.

Example 60: The sensor chip according to any one of the preceding examples, wherein the sensor chip and/or the reaction cell has an elongated shape; and wherein the membrane is arranged at a longitudinal side of the sensor chip.

Example 61: The sensor chip according to any one of the preceding examples, wherein the reaction cell has a rectangular cross-section; and/or wherein the reaction cell is formed as parallelepiped.

Example 62: The sensor chip according to any one of the preceding examples, wherein the reaction cell has a circular, elliptical or oval cross-section; and/or wherein the reaction cell is tubular shaped.

Example 63: The sensor chip according to any one of the preceding examples, wherein the membrane at least partly encompasses the reaction cell along a perimeter of the reaction cell.

Example 64: The sensor chip according to any one of the preceding examples, further including:
at least one reservoir fluidly couplable with the reaction cell, wherein the at least one reservoir is configured to supply de-ionized water to the reaction cell.

Example 65: The sensor chip according to example 64, wherein the at least one reservoir is at least partly filled with de-ionized water.

Example 66: The sensor chip according to any one of examples 64 to 65, wherein at least a part of a wall of the at least one reservoir is displaceable, flexible or movable, such that a volume of the reservoir is adjustable.

Example 67: The sensor chip according to any one of examples 64 to 66, wherein at least a part of the at least one reservoir is formed as flexible bag or flexible blister.

Example 68: The sensor chip according to any one of examples 64 to 67, further including:
at least one movable piston configured to adjust a volume of the at least one reservoir.

Example 69: The sensor chip according to any one of examples 64 to 68, wherein the at least one reservoir is encompassed by a portion of a housing of the sensor chip; and wherein the portion of the housing comprises at least one opening for pressure equalization.

Example 70: The sensor chip according to any one of examples 64 to 69, wherein the at least one reservoir is fluidly coupled to the reaction cell by a semi-permeable membrane blocking salts to diffuse from the reaction cell into the reservoir.

Example 71: The sensor chip according to any one of examples 64 to 70, further including:
at least one blocking element configured to block fluid communication between the reaction cell and the at least one reservoir.

Example 72: The sensor chip according to example 71, wherein the sensor chip is activatable based on unblocking fluid communication between the reaction cell and the at least one reservoir using the at least one blocking element.

Example 73: The sensor chip according to any one of examples 71 to 72, wherein the at least one blocking element comprises a water-impermeable membrane arranged between the at least one reservoir and the reaction cell.

Example 74: The sensor chip according to example 73, wherein the sensor chip is activatable based on disrupting at least a part of the water-impermeable membrane.

Example 75: The sensor chip according to any one of examples 71 to 74, wherein the at least one blocking element comprises a movable pin operable to block or unblock fluid communication between the reaction cell and the at least one reservoir.

Example 76: The sensor chip according to example 75, wherein the sensor chip is activatable based on displacing the movable pin, such that fluid communication between the reaction cell and the at least one reservoir is unblocked.

Example 77: The sensor chip according to any one of the preceding examples, wherein the receptor protein complexes each comprise at least one ligand binding domain of a receptor protein configured to bind to the one or more bioactive agents and configured to change a state and/or conformation upon binding to one of the bioactive agents.

Example 78: The sensor chip according to any one of the preceding examples, wherein at least some of the receptor protein complexes comprise a plurality of ligand binding domains of one or more receptor proteins.

Example 79: The sensor chip according to any one of the preceding examples, wherein at least some of the receptor protein complexes comprise a plurality of ligand binding domains of different type configured to bind to different types of bioactive agents.

Example 80: The sensor chip according to any one of examples 76 to 78, wherein the receptor protein is a xenosensor protein or a hormone receptor protein.

Example 81: The sensor chip according to any one of the preceding examples, wherein the receptor protein complexes each comprise at least one ligand binding domain of a receptor protein selected from the group consisting of Aryl Hydrocarbon Receptor, Constitutive Androstane Receptor, Pregnane X Receptor, Estrogen Receptor, and aryl hydrocarbon receptor.

Example 82: The sensor chip according to any one of the preceding examples, wherein at least a part of the receptor protein complexes comprises a recombinant protein or a non-recombinant protein.

Example 83: The sensor chip according to any one of the preceding examples, wherein the receptor protein complexes each comprise at least a part of a monomer receptor protein, a homodimer receptor protein complex or a heterodimer receptor protein complex.

Example 84: The sensor chip according to any one of the preceding examples, wherein the receptor protein complexes comprise at least a part of different types of receptor proteins configured to bind to different types of bioactive agents.

Example 85: The sensor chip according to any one of the preceding examples, wherein at least some of the receptor protein complexes comprise an organic or inorganic moiety dissociating from a remaining part of the respective receptor protein complex upon binding to the one or more bioactive agents.

Example 86: The sensor chip according to any one of the preceding examples, wherein the receptor protein complexes comprise at least one fluorescent label dissociating from a remaining part of the respective receptor protein complex upon binding to the one or more bioactive agents.

Example 87: The sensor chip according to any one of the preceding examples, wherein at least some of the receptor protein complexes comprise a plurality of fluorescent labels of same or different type.

Example 88: The sensor chip according to any one of examples 85 to 86, wherein the at least one fluorescent label is a fluorescent dye or green fluorescent protein.

Example 89: The sensor chip according to any one of the preceding examples, wherein the receptor protein complexes comprise a nanoparticle dissociating from a remaining part of the respective receptor protein complex upon binding to the one or more bioactive agents.

Example 90: The sensor chip according to any one of the preceding examples, wherein the receptor protein complexes comprise an affinity tag configured to bind to a molecular trapping complex arranged within the reaction cell.

Example 91: The sensor chip according to any one of the preceding examples, further including:
a chip identifier for identifying a type of the sensor chip and/or a type of receptor protein complex contained in the sensor chip.

Example 92: The sensor chip according to example 91, wherein the chip identifier is readable by a user device and/or readable by the sensing device.

Example 93: The sensor chip according to any one of examples 91 to 92, wherein the chip identifier contains information or data indicative of a type of the sensor chip and/or a type of receptor protein complex contained in the sensor chip.

Example 94: The sensor chip according to any one of the preceding examples, wherein the chip identifier includes one or more of a barcode, a QR code, an RFID tag, a label, and a data storage.

Example 95: The sensor chip according to any one of the preceding examples, further including:
a data storage configured to store historic data indicative of a usage or remaining lifetime of the sensor chip.

Example 96: The sensor chip according to example 95, wherein the data storage is configured to store one or more operational parameters indicative of the usage or remaining lifetime of the sensor chip.

Example 97: The sensor chip according to any one of examples 95 to 96, wherein the data storage is accessible by the sensing device upon operatively coupling the sensor chip to the sensing device.

Example 98: The sensor chip according to any one of the preceding examples, wherein the sensor chip includes a plurality of reaction cells.

Example 99: The sensor chip according to any one of the preceding examples, wherein the plurality of reaction cells comprise different types of receptor protein complexes configured to bind to different types of bioactive agents.

Example 100: The sensor chip according to any one of the preceding examples, further including:
one or more sensors configured to determine the state change of the at least part of the receptor protein complexes.

Example 101: The sensor chip according to any one of the preceding examples, further including:
processing circuitry coupled with the one or more sensors and configured to provide a detection signal indicative of a determined state change of the at least part of the receptor protein complexes.

Example 102: Use of the sensor chip according to any one of the preceding examples for detecting one or more bioactive agents.

Example 103: A sensing device operatively couplable to at least one sensor chip according to any one of examples 1 to 101 for detecting one or more bioactive agents in a surrounding medium, the sensing device comprising:
at least one sensor configured to determine a state change of at least a part of the receptor protein complexes in a reaction cell of the at least one sensor chip, the state change being induced by binding of one or more receptor protein complexes with one or more bioactive agents entering from the surrounding medium through a membrane of the sensor chip into the reaction cell of the sensor chip; and
processing circuitry coupled with the at least one sensor, wherein the processing circuitry is configured to provide a detection signal indicative of presence of the one or more bioactive agents in the surrounding medium based on determining the state change of the at least part of the receptor protein complexes.

Example 104: A sensing device for detecting one or more bioactive agents in a surrounding medium, the sensing device comprising:
a housing configured to at least partly receive at least one senor chip according to any one of examples 1 to 101 to operatively couple the sensing device to the at least one sensor chip; and
processing circuitry configured to provide a detection signal indicative of presence of the one or more bioactive agents in the surrounding medium based on determining a state change of at least a part of the receptor protein complexes in a reaction cell of the at least one sensor chip, the state change being induced by binding of said one or more receptor protein complexes with one or more bioactive agents entering from the surrounding medium through a membrane of the sensor chip into the reaction cell of the sensor chip.

Example 105: The sensing device according to any one of examples 103 and 104, wherein the surrounding medium includes environmental air; and/or
wherein the sensing device is an environmental monitoring device for monitoring a quality of environmental air.

Example 106: The sensing device according to any one of examples 103 to 105, wherein the sensing device is a stationary device and/or desktop device.

Example 107: The sensing device according to any one of examples 103 to 105, wherein the sensing device is a portable device, a mobile device and/or a handheld device.

Example 108: The sensing device according to any one of examples 103 to 107, wherein the detection signal is an electronically processable signal.

Example 109: The sensing device according to any one of examples 103 to 108, wherein the detection signal is indicative of an amount of one or more bioactive agents per volume of surrounding medium, a mass of bioactive agent per volume of surrounding medium, a concentration of bioactive agent in the surrounding medium, a receptor protein activation potential of the surrounding medium, and a bioactivity of the surrounding medium.

Example 110: The sensing device according to any one of examples 103 to 109, wherein the sensing device is configured to at least partly receive the at least one sensor chip.

Example 111: The sensing device according to any one of examples 103 to 110, wherein the sensing device is mechanically couplable to the sensor chip.

Example 112: The sensing device according to any one of examples 103 to 111, wherein the sensing device is magnetically couplable to the sensor chip.

Example 113: The sensing device according to any one of examples 103 to 112, wherein the sensing device comprises at least one socket configured to at least partly receive the at least one sensor chip.

Example 114: The sensing device according to example 113, wherein the at least one socket is configured to at least partly receive the at least one sensor chip, such that a housing of the sensing device at least partly encompasses the at least one sensor chip.

Example 115: The sensing device according to any one of examples 113 to 114, wherein the at least one socket comprises at least one surface feature formed complementary to at least one surface feature of the sensor chip to ensure correct positioning of the sensor chip in the at least one socket.

Example 116: The sensing device according to example 115, wherein the at least one surface feature of the at least one socket includes one or more guides for positioning the sensor chip within the socket.

Example 117: The sensing device according to any one of examples 115 to 116, wherein the at least one surface feature of the at least one socket is configured to at least partly engage with the at least one surface feature of the sensor chip, such that the sensor chip is removably fixable in the socket.

Example 118: The sensing device according to any one of examples 103 to 117, wherein the sensing device comprises a plurality of sockets configured to receive a plurality of sensor chips of same or different type.

Example 119: The sensing device according to any one of examples 103 to 118, wherein the sensing device includes one or more magnets for magnetically coupling the sensor chip to the sensing device.

Example 120: The sensing device according to any one of examples 103 to 119, wherein the sensing device is configured to determine the state change of the at least part of the receptor protein complexes based on an optical measurement, based on detecting fluorescent light, based on fluorescent excitation of one or more components of the receptor protein complexes, based on light scattering, based on determining a conductivity of a substrate contained in the reaction cell of the sensor chip, based on a electrochemical process occurring in the reaction cell, based on determining one or more optical properties of at least one functional surface of the reaction cell, based on determining absorption of electromagnetic radiation, based on determining a mass of at least one functional surface of the reaction cell, based on determining a mass of receptor protein complexes confined to or immobilized at at least one functional surface of the reaction cell, and based on surface plasmon resonance at at least one functional surface of the reaction cell.

Example 121: The sensing device according to any one of examples 103 to 120, wherein the processing circuitry is configured to determine presence of the one or more bioactive agents in the surrounding medium based on detecting, with at least one sensor of the sensor chip or the sensing device, a positional change of one or more receptor proteins within the reaction cell upon binding to one or more of the bioactive agents.

Example 122: The sensing device according to any one of examples 103 to 121, wherein the sensing device is configured to determine one or more of a presence, a number, a mass, a density, and a mass density of receptor protein complexes dissolved in a substrate of the reaction cell of the at least one sensor chip; and/or
wherein the sensing device is configured to determine one or more of a presence, a number, a mass, a density, and a mass density of receptor protein complexes dissolved in a substrate of the reaction cell of the at least one sensor chip based on determining a change in at least one of an optical and an electrical signal transmitted to the sensor chip.

Example 123: The sensing device according to any one of examples 103 to 122, wherein the receptor protein complexes are configured to dissociate from at least one functional surface of the reaction cell upon binding to one of the bioactive agents; and
wherein the sensing device is configured to determine one or more of a number, a mass, a density, and a mass density of receptor protein complexes immobilized at or liberated from at least one functional surface of the reaction cell.

Example 124: The sensing device according to any one of examples 103 to 123, wherein the sensing device includes one or more piezo elements configured to determine a mass of receptor protein complexes immobilized at or liberated from at least one functional surface of the reaction cell to detect the one or more bioactive agents in the surrounding medium.

Example 125: The sensing device according to example 124, wherein the one or more piezo elements are configured to determine the mass of receptor protein complexes immobilized at or liberated from the at least one functional surface of the reaction cell based on determining a mass of one or more components of the receptor protein complexes bound to an inner surface of the reaction cell disposed towards the at least one functional surface of the reaction cell.

Example 126: The sensing device according to any one of examples 124 to 125, wherein the one or more piezo elements are mechanically couplable to one or more functional surfaces of the reaction cell of the sensor chip; and/or
wherein the one or more piezo elements are mechanically couplable to one or more inner surfaces of the reaction cell of the sensor chip disposed towards one or more functional surfaces of the reaction cell.

Example 127: The sensing device according to any one of examples 103 to 124, wherein the receptor protein complexes comprise at least one fluorescent label; and
wherein the sensing device is configured to detect the one or more bioactive agents based on exciting the one or more fluorescent labels and based on detecting fluorescence light emitted by the one or more fluorescent labels.

Example 128: The sensing device according to any one of examples 103 to 127, wherein the receptor protein complexes comprise at least one fluorescent label dissociating from a remaining part of the respective receptor protein complex upon binding to one or more of the bioactive agents; and
wherein the sensing device is configured to detect the one or more bioactive agents based on exciting one or more fluorescent labels dissociated from one or more receptor protein complexes and based on detecting fluorescence light emitted by the one or more dissociated fluorescent labels.

Example 129: The sensing device according to any one of examples 127 to 128, wherein the sensing device includes one or more light sources configured to excite the one or more fluorescent labels; and
wherein the sensing device includes one or more photodetectors configured to detect fluorescence light emitted by the one or more fluorescent labels.

Example 130: The sensing device according to any one of examples 103 to 129, wherein the sensing device includes one or more light sources configured to illuminate at least a part of the reaction cell; and
wherein the sensing device includes one or more photodetectors configured to detect light scattered by one or more components of the receptor protein complexes within the reaction cell.

Example 131: The sensing device according to any one of 129 to 130, wherein the one or more photodetectors are arranged adjacent to a socket of the sensing device for at least partly receiving the sensor chip to detect electromagnetic radiation penetrating out of the reaction cell.

Example 132: The sensing device according to any one of examples 129 to 131, wherein the one or more photodetectors are arranged to oppose a detection window of the sensor chip.

Example 133: The sensing device according to any one of examples 129 to 132, wherein the one or more light sources are arranged adjacent to a socket of the sensing device for at least partly receiving the sensor chip, such that electromagnetic radiation emitted by the one or more light sources is couplable into the reaction cell.

Example 134: The sensing device according to any one of examples 129 to 133, wherein the one or more light sources are arranged to couple electromagnetic radiation through one or more optical connectors of the sensor chip into the reaction cell.

Example 135: The sensing device according to any one of examples 103 to 134, wherein the sensing device includes one or more light sources arranged to couple electromagnetic radiation into one or more optical guides traversing at least a part of the reaction cell of the sensor chip; and
wherein the sensing device includes one or more photodetectors configured to detect electromagnetic radiation traversing the one or more optical guides.

Example 136: The sensing device according to example 135, wherein the one or more light sources are arranged to align with an end of the one or more optical guides; and/or
wherein the one or more photodetectors are arranged to align with an end of the one or more optical guides.

Example 137: The sensing device according to any one of examples 103 to 136, wherein the processing circuitry is configured to determine one or more optical properties of at least one of a functional surface and an inner surface of the reaction cell arranged adjacent the one or more optical guides.

Example 138: The sensing device according to any one of examples 135 to 137, wherein the processing circuitry is configured to determine the one or more optical properties based on determining absorption of evanescence light penetrating through the one or more optical guides into the reaction cell.

Example 137: The sensing device according to any one of examples 103 and 105 to 136, further including:
a housing configured to at least partly encompass the sensor chip;
wherein the housing includes one or more holes for passing surrounding medium therethrough, such that at least a part of the sensor chip is contactable with the surrounding medium.

Example 140: The sensing device according to example 139, further including one or more channels for passing surrounding medium through the one or more holes in the housing of the sensing device to said at least part of the sensor chip.

Example 141: The sensing device according to example 140, further including one or more ventilation devices arranged in the one or more channels and configured to convey the surrounding medium through the one or more holes in the housing of the sensing device to said at least part of the sensor chip.

Example 142: The sensing device according to any one of examples 103 to 141, further including one or more heating elements configured to heat at least a part of the sensor chip.

Example 143: The sensing device according to example 142, wherein the one or more heating elements are arranged adjacent to a socket of the sensing device for at least partly receiving the sensor chip.

Example 144: The sensing device according to any one of examples 103 to 143, further including one or more temperature sensors configured to determine a temperature of at least a part of the sensor chip.

Example 145: The sensing device according to example 144, wherein the processing circuitry is configured to control a temperature of at least a part of the sensor chip based on determining a temperature of said at least part of the sensor chip using the one or more temperature sensors.

Example 146: The sensing device according to any one of examples 103 to 145, further including:
one or more electrical connectors for connecting to one or more electrical connectors of the sensor chip upon insertion of the sensor chip into the sensing device.

Example 147: The sensing device according to any one of examples 103 to 146, wherein the processing circuitry is couplable to one or more electrodes of the sensor chip; and
wherein the processing circuitry is configured to determine a conductivity of a substrate or composition within the reaction cell.

Example 148: The sensing device according to any one of examples 103 to 147, wherein the processing circuitry is configured to determine one or more operational parameters indicative of a usage or remaining lifetime of the sensor chip; and/or
wherein the processing circuitry is configured to determine a usage or a remaining lifetime of the at least sensor chip based on determining one or more operational parameters of the sensor chip.

Example 149: The sensing device according to example 148, wherein the processing circuitry is configured to determine the usage or remaining lifetime of the at least one sensor chip based on retrieving historic data indicative of the usage or remaining lifetime of the sensor chip from a data storage of the sensor chip.

Example 150: The sensing device according to any one of examples 148 to 149, wherein the processing circuitry is configured to determine the usage or remaining lifetime of the at least one sensor chip based on comparing the determined one or more operational parameters of the sensor chip with one or more threshold values.

Example 151: The sensing device according to any one of examples 144 to 150, wherein the processing circuitry is configured to determine the usage or remaining lifetime of the at least one sensor chip based on determining a type of the sensor chip and/or a type of receptor protein complexes employed in the sensor chip.

Example 152: The sensing device according to any one of examples 148 to 151, wherein the processing circuitry is configured to store the one or more operational parameters in a data storage of the sensing device and/or a data storage of the sensor chip.

Example 153: The sensing device according to any one of examples 148 to 152, wherein the processing circuitry is configured to determine the one or more operational parameters based on monitoring a temperature of at least a part of the sensor chip.

Example 154: The sensing device according to any one of examples 103 to 153, wherein the processing circuitry is configured to determine depletion of receptor protein complexes in the sensor chip over time.

Example 155: The sensing device according to any one of examples 103 to 154, wherein the processing circuitry is configured to determine a remaining lifetime of the sensor chip based on determining depletion of receptor protein complexes in the sensor chip over time.

Example 156: The sensing device according to any one of examples 103 to 155, wherein the sensing device includes a user interface; and
wherein the processing circuitry is configured to provide a notification to a user via the user interface upon expiry of a lifetime of the sensor chip.

Example 157: The sensing device according to any one of examples 103 to 156, wherein the sensing device includes a user interface; and wherein the processing circuitry is configured to provide information related to the determined one or more bioactive agents to a user via the user interface.

Example 158: The sensing device according to any one of examples 103 to 157, wherein the sensing device includes a user interface; and
wherein the processing circuitry is configured to notify the user via the user interface upon determining a concentration of bioactive agents in the surrounding medium reaching or exceeding a predefined threshold.

Example 159: The sensing device according to any one of examples 156 to 158, wherein the user interface includes one or more of a vibrational element, a display, one or more LEDs, and a speaker.

Example 160: The sensing device according to any one of examples 103 to 159, wherein the sensor chip includes a chip identifier for identifying a type of the sensor chip and/or a type of receptor protein complex contained in the sensor chip; and
wherein the processing circuitry is configured to retrieve information or data indicative of a type of the sensor chip and/or a type of receptor protein complex contained in the sensor chip from the chip identifier.

Example 161: The sensing device according to any one of examples 103 to 160, wherein the sensing device includes one or more of a barcode reader, a QR code reader, and an RFID scanner.

Example 162: The sensing device according to any one of examples 103 to 161, further including one or more energy storages for supplying electrical energy.

Example 163: The sensing device according to any one of examples 103 to 162, further including communication circuitry for communicatively coupling the sensing device with a user device.

Example 164: The sensing device according to example 163, wherein the communication circuitry includes a wireless communication circuitry, preferably a Bluetooth transmitter.

Example 165: The sensing device according to any one of examples 103 to 164, further including one or more radiation sensor for measuring exposure of the sensing device to ionizing radiation.

Example 166: The sensing device according to example 165, wherein the one or more radiation sensors include at least one of a reversibly photochromic layer sensitive to UV radiation and a radiochromic dye film sensitive to ionizing radiation.

Example 167: The sensing device according to any one of examples 103 to 166, further including one or more of a humidity sensor, a Volatile Organic Carbon sensor, an Ozone sensor, a particulate matter sensor, a sensor for nitric acid, and a carbon monoxide sensor.

Example 168: Use of a sensing device to any one of examples 103 to 167 for detecting one or more bioactive agents in a surrounding medium.

Example 169: A sensing system for detecting one or more bioactive agents in a surrounding medium, the system comprising:
at least one sensor chip according to any one of examples 1 to 101; and
a sensing device according to any one of examples 103 to 167.

Example 170: A method of detecting one or more bioactive agents in a surrounding medium with a sensing system according to example 169, the method comprising:
passing one or more bioactive agents through a membrane of the sensor chip into a reaction cell of the sensor chip;
binding one or more bioactive agents to one or more receptor protein complexes, thereby inducing a state change of at least a part of the receptor protein complexes;
detecting, with the sensing device, the induced state change of said at least a part of the receptor protein complexes; and
generating a detection signal indicative of presence of the one or more bioactive agents in the surrounding medium based on the determined state change of said at least a part of the receptor protein complexes.

Example 172: The method according to example 170, wherein passing one or more bioactive agents through the membrane of the sensor chip includes contacting a membrane of the sensor chip with the surrounding medium.

Example 172: The method according to any one of examples 170 to 171, further including:
activating the sensor chip based on supplying de-ionized water from a reservoir of the sensor chip to the reaction cell of the sensor chip.

Example 173: The method according to example 172, wherein activating the sensor chip includes unblocking fluid communication between the reaction cell and the at least one reservoir using at least one blocking element between the reservoir and the reaction cell of the sensor chip.

Example 174: The method according to example 173, wherein the at least one blocking element includes a water-impermeable membrane arranged between the reservoir and the reaction cell; and
wherein activating the sensor chip includes disrupting at least a part of the water-impermeable membrane.

Example 175: The method according to any one of examples 173 to 174, wherein the at least one blocking element includes a movable pin arranged between the reservoir and the reaction cell; and
wherein activating the sensor chip includes displacing the movable pin.

Example 176: The method according to any one of examples 173 to 175, wherein activating the sensor chip includes removing a sealing cover from an outer surface of the membrane.

Example 177: The method according to any one of examples 170 to 176, wherein detecting the induced state change includes determining one or more of a number, a mass, a density, and a mass density of receptor protein complexes dissolved in a substrate of the reaction cell of the at least one sensor chip.

Example 178: The method according to any one of examples 170 to 177, wherein detecting the induced state change includes determining one or more of a number, a mass, a density, and a mass density of receptor protein complexes immobilized at or liberated from at least one functional surface of the reaction cell.

Example 179: The method according to any one of examples 170 to 178, wherein detecting the induced state includes determining one or more of a number, a mass, a density, and a mass density of receptor protein complexes immobilized at or liberated from at least one inner surface of the reaction cell disposed towards at least one functional surface of the reaction cell.

Example 180: The method according to any one of examples 170 to 179, wherein detecting the induced state change includes exciting one or more fluorescent labels of the receptor protein complexes and detecting fluorescence light emitted by the one or more fluorescent labels.

Example 181: The method according to any one of examples 170 to 180, wherein detecting the induced state change includes detecting light scattered by one or more components of the receptor protein complexes within the reaction cell.

Example 182: The method according to any one of examples 170 to 181, wherein detecting the induced state change includes determining one or more optical properties of at least one of a functional surface and an inner surface of the reaction cell.

Example 183: The method according to any one of examples 170 to 182, wherein detecting the induced state change includes determining absorption of evanescence light penetrating through one or more optical guides of the sensor chip into the reaction cell.

Examples will now be further described with reference to the Figures in which:

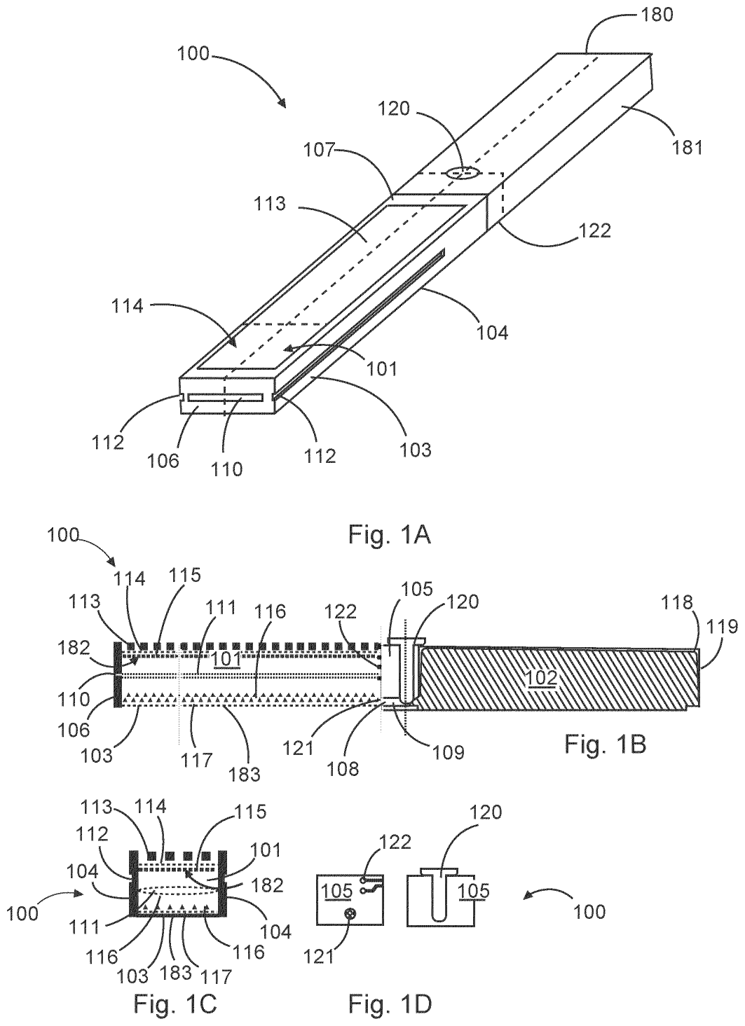

FIG. 1A shows a perspective view of a sensor chip for detecting one or more bioactive agents;

FIGS. 1B, 1C and 1D each show a cross-sectional view of the sensor chip of FIG. 1A;

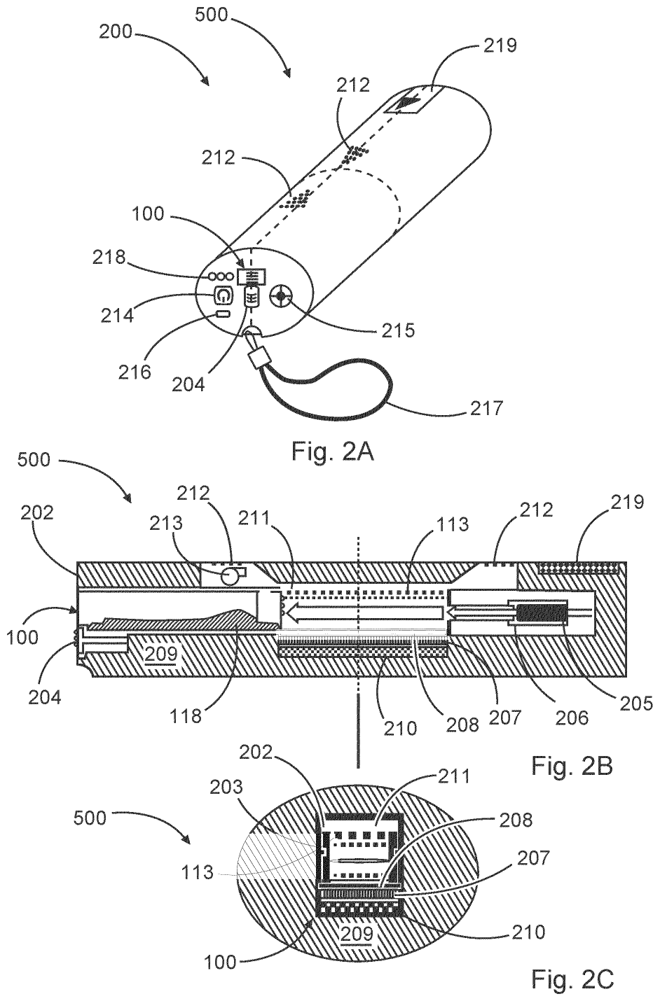

FIG. 2A shows a perspective view of a sensing system for detecting one or more bioactive agents;

FIG. 2B shows a cross-sectional view of the sensing system of FIG. 2A;

FIG. 2C shows another cross-sectional view of the sensing system of FIG. 2A;

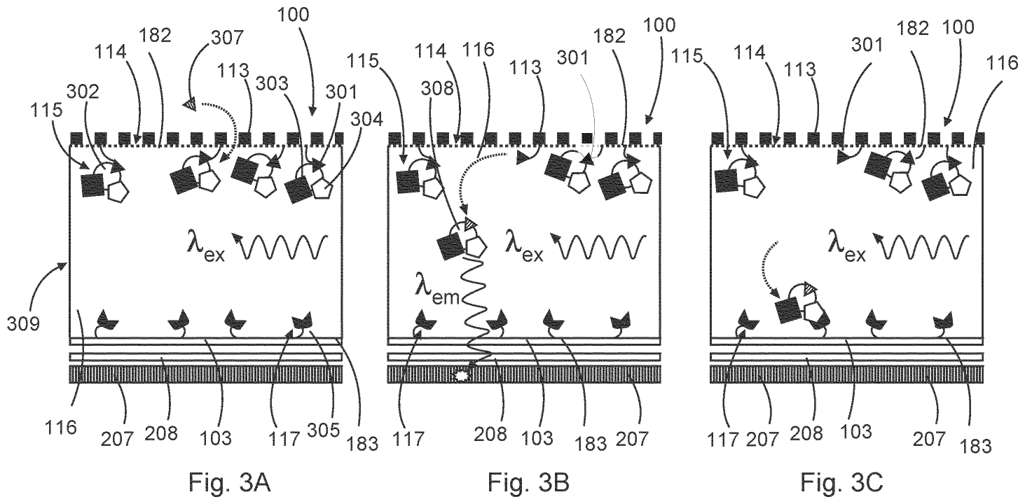

FIGS. 3A to 3C each show a cross-sectional view of a reaction cell of a sensor chip;

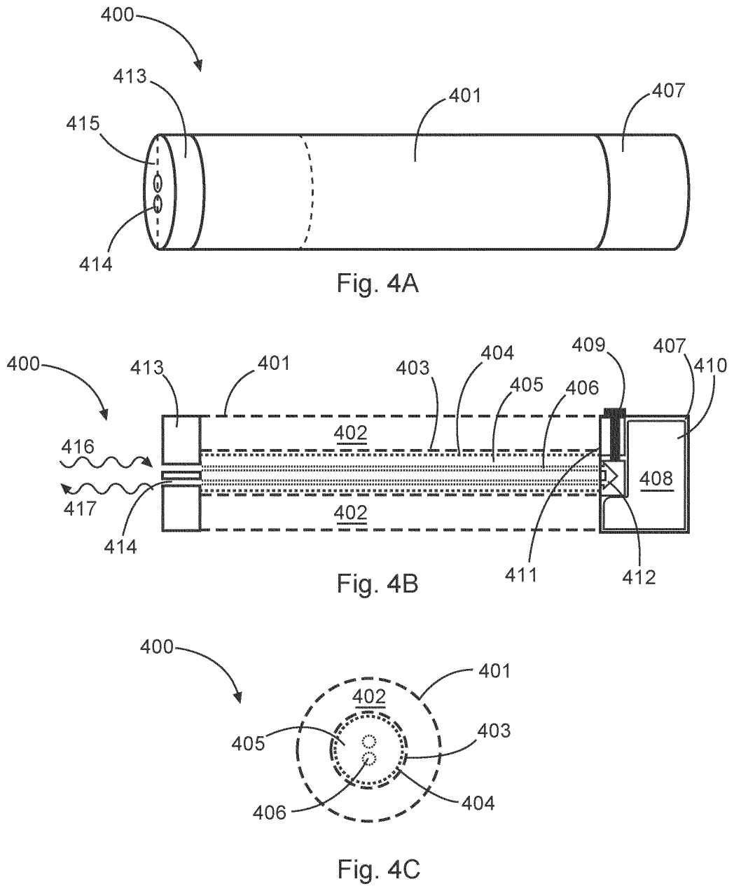

FIG. 4A shows a perspective view of a sensor chip for detecting one or more bioactive agents;

FIGS. 4B and 4C each show a cross-sectional view of the sensor chip of FIG. 4A;

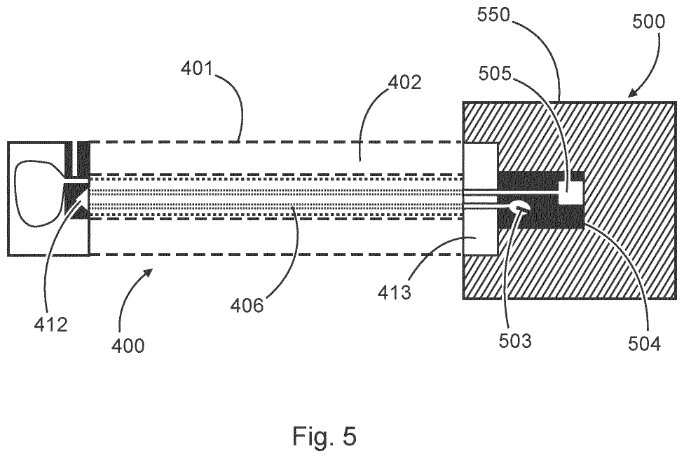

FIG. 5 shows a cross-sectional view of a sensing system for detecting one or more bioactive agents;

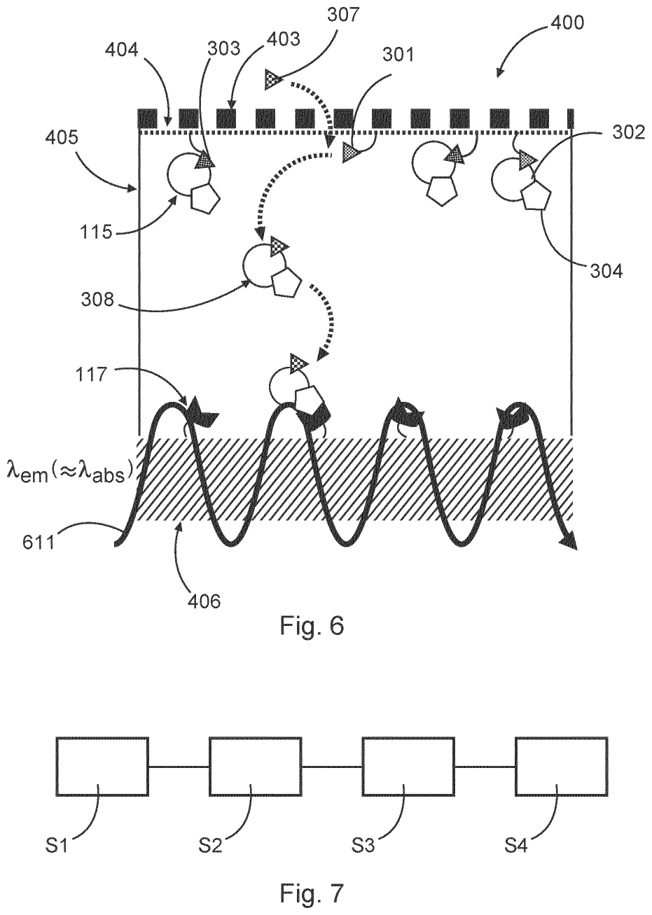

FIG. 6 shows a cross-sectional view of a reaction cell of a sensor chip;

FIG. 7 shows a flowchart illustrating a method of detecting one or more bioactive agents in a surrounding medium.

The Figures are schematic only and not true to scale. In principle, identical or like parts, elements and/or steps are provided with identical or like reference numerals in the figures.

FIG. 1A shows a perspective view of a sensor chip 100 for detecting one or more bioactive agents. FIGS. 1B, 1C and 1D show cross-sectional views of the sensor chip 100 of FIG. 1A.

The sensor chip 100 shown of FIGS. 1A to 1D is exemplary shown or designed as an elongated structure with rectangular cross-section, for example in a projection parallel to the longitudinal axis 180 of the sensor chip 100.

The sensor chip 100 comprises a housing 181 or an outer housing 181, which encloses a reaction cell 101. The sensor chip 100 further comprises an open grid 113 beneath which a membrane 114 separating the reaction cell 101 from a surrounding medium, such as air or water is located. The reaction cell 101 includes receptor protein complexes 115 configured to bind to one or more bioactive agents in the reaction cell 101, such that a detectable state change of at least a part of the receptor protein complexes 115 is induced, as described hereinabove and as will be described in further detail hereinbelow.

Optionally, the sensor chip 100 includes a reservoir 102 or water reservoir 102, which may be integrally formed with the housing 181. Accordingly, the part of the housing 181 forming the water reservoir 102 can be made of only one single material, whereas the part of the housing 181, where the reaction cell 101 is located, can be divided into several parts or portions. In particular, the sensor chip 100 or its housing 181 comprises a light permeable lower part 103 or detection window 103, two parts 104 flanking the light permeable part 103, a rectangular part 105 facing the reservoir 102, another rectangular wall 106 located on the opposite side and a flat rectangular part 107 on the upper side. The rectangular part 107 on the upper side comprises the open grid 113.

The light permeable part 103 or detection window 103 of the sensor chip 100 can be made of a material that is permeable to light of a specified wavelength λₑₘ, for example an emission wavelength of fluorescent emission, but optionally not permeable to light of another specified wavelength λₑₓ, for example an excitation wavelength for fluorescence excitation.

The parts 104, 105 and 106 of the housing are not permeable to light and may optionally be coated on the side facing the inner volume of the reaction cell 101 with a material of low protein adsorptivity, for instance fluoropolymers.

Part 105 of the housing 181 provides an aperture 108 which can hold channel 109, for example a flexible micro-channel 109.

Part 106 of the housing 181 comprises an optical connector or aperture 110 which can be sealed by a material permeable to light of a specified wavelength λₑₓ, such as an excitation wavelength for fluorescence excitation.

The aperture 110 or optical connector 110 preferably has a shape such that light passing therethrough results in a sheath of light 111 that spans the width of the reaction cell 101. The optical connector 110 or aperture 110 is preferably located at a position that results in the sheath 111 of light being located in immediate proximity to the gas permeable membrane, 114 preferably within a distance of 0.1 mm or less.

The flanking parts 104 of the housing 181 provide one or more surface features 112, such as guides 112, that allow mounting the sensor chip 100 in a sensing device and assure proper orientation and positioning.

The flat rectangular part 107 of the housing 181 comprises an open grid 113, for example with a mesh size in the range of about 0.2-2 mm. Directly underneath the grid 113 and preferably fused with the grid 113, the gas-permeable membrane 114 is arranged, such that the membrane 114 is protected.

The membrane 114 is of little thickness, low diffusion resistance and of low protein adsorptivity. The inner side of the permeable membrane 114 directly faces the inner volume of the reaction cell 101.

When the sensor chip 100 is not mounted in the sensing device, the open grid 113 can be covered and sealed air-tight by a removable sealing cover (not shown), such as a foil. When removing the sensor chip 100 from the sensing device again, for instance because the sensor chip 100 is temporarily not used or because the user intends to change the type of sensor chip 100, for example to focus on another aspect of environmental quality, this sealing cover can be put on the grid 113 again and will keep water from evaporating from the reaction cell 101 and receptor protein complexes 115 be consumed.

Within the reaction cell 101, receptor protein complexes 115 are immobilized at the gas-permeable membrane 114. The receptor protein complexes 115 can particularly be immobilized at a functional surface 182 of the reaction cell 101, which in the example of FIGS. 1A to 1D is defined by an inner surface of the membrane 114 facing the interior of the reaction cell 101.

Immobilization of the receptor protein complexes at the functional surface 182 or inner surface of the membrane 114 can be achieved by covalently linked molecules that are known, low affinity ligands of the receptor protein complexes 115.

The reaction cell 101 can contain a preferably lyophilized substrate 116, for example a mixture 116 of salts, a buffer and, optionally, proteins that stabilize the receptor protein complexes 115 and/or low amounts of a surface-active molecule, such as tween or triton X-100.

The light permeable wall 103 or detection window 103 can optionally be coated with covalently linked molecules or molecular trapping complexes 117 that are known to be high affinity ligands of the receptor protein complexes 115 or binding a part of the receptor protein complexes 115 with high affinity. These molecular trapping complexes 117 can act as a sink for liberated receptor protein complexes 115 or parts thereof and decrease the incidence of multiple signals generated by the same liberated receptor protein complex 115. The molecular trapping complexes 117 can be covalently linked to an inner surface 183 of the reaction cell 101, which inner surface 183 is disposed towards the functional surface 182 or membrane 114. In the example of FIGS. 1A to 1D, the inner surface 183 of the reaction cell 101 is arranged opposite to the functional surface 182 and/or the membrane 114.

Molecules or the crosslinker that binds the molecular trapping complexes 117 to the inner surface of the detection window 103 may not absorb light of the specified or predefined wavelength λₑₘ, for example the emission wavelength of fluorescence emission. In addition, molecules known to quench fluorescence of the predefined wavelength λₑₘ may be linked covalently to the inner surface of the detection window 103 (not shown).

The water reservoir 102 may have an adjustable volume. For instance the reservoir 102 may include or may be defined by a flexible blister 118 containing de-ionized water. The flexible blister 118 is connected to the reaction cell 101 via a channel 109, such as a flexible microchannel 109 of for example 0.1-0.5 mm diameter. The channel 109 is essentially an extension of the blister 118, reaching through the aperture 108. The part of the housing 181, in which the flexible blister 118 is contained, comprises one or several small holes 119 or openings 119 through which air or surrounding medium may enter the chamber or reservoir 102 allowing for water efflux from the blister 118 without generation of a negative pressure in the chamber or reservoir 102.

The channel 109 can be obstructed by a blocking element 120, for example a movable or removable pin 120. In addition, the reaction cell 101 and the de-ionized water are separated by a semi-permeable membrane 121 present at the end of the channel 109, for example directly facing the reaction cell 101, which, in absence of the removable pin 120, prevents mixing of the water in the blister 118 with the buffered isotonic solution in the reaction cell 101.

In the unused sensor chip 100, the deionized water contained in the flexible blister 118 is preferably stored at over-pressure relative to the surrounding. Specifically, in the unused state of the chip 100, the flexible blister 118 can be slightly stretched and, upon opening the channel 109 to the reaction cell 101, actively pushes an initial amount of the de-ionized water into the reaction cell 101.

On the side facing the reaction cell 101, part 105 of the housing 180 of the sensor chip 100 comprises electrodes 122 that allow measuring the conductivity of the de-ionized water in which the substrate 116 or matrix 116 inside the reaction cell 101 is dissolved. Conductivity measurements may provide an effective means for monitoring the osmolarity of the solution present in the reaction cell 101, which may be indicative for whether the protein complexes 115 are in their native, functional state.

FIG. 2A shows a perspective view of a sensing system 500 for detecting one or more bioactive agents. FIGS. 2B and 2C each show a cross-sectional view of the sensing system 500 of FIG. 2A. In particular, FIGS. 2A to 2C show a sensing system 500 with a sensing device 200 and a sensor chip 100 inserted into the sensing device 200. Unless stated otherwise, the sensor chip 100 of FIGS. 2A to 2C comprises the same features as the sensor chip 100 described with reference to FIGS. 1A to 1D.

In particular, FIGS. 2A to 2C show the sensing device 200 with the sensor chip 100 being installed, coupled or mounted thereto. The device 200 comprises a socket 202, into which the sensor chip 100 can be inserted axially. The socket 202 comprises surface features 203, such as guides 203, that ensure proper orientation and positioning of the sensor chip 100, and optionally a sensor (not shown) detecting whether a sensor chip 100 is inserted and whether it is properly oriented. This sensor can deactivate the sensing device 200 if no chip is inserted or if it is inserted improperly.

Once inserted, the sensor chip 100 is held in place by a click mechanism 204, for example a hook that is pushed towards the sensor chip 100 by a spring. Mechanisms such as magnetic connections may be used instead or in addition.

The sensing device 200 furthermore provides a light source 205, for instance a laser diode, which, when the device 200 is active, emits light of a specified wavelength λₑₓ, or a broader range of wavelengths covering λₑₓ, such as a range of fluorescence excitation wavelengths. The light source 205 is mounted in a housing 206 of the sensing device 200 and is located so that it emits light through the optical connector 110 or aperture 110 of the sensor chip 100 into the reaction cell 101 of the sensor chip 100.

The sensing device 200 furthermore provides one or more sensors 207 for detecting the state change induced by binding of the receptor protein complex 115 with one or more bioactive agents in the reaction cell 101.

In the example shown in FIGS. 2A to 2C, the sensing device 200 includes one or more photosensors 207, for example an array of photosensors 207. These are located below the light permeable part 103 or detection window 103 of the sensor chip 100 and are optionally protected by a layer 208 of a material permeable to light of a specified wavelength λₑₘ or range of wavelengths, such as a range of fluorescence emission wavelengths.

The sensing device 200 further comprises processing circuitry 209 coupled with the at least one sensor 207, wherein the processing circuitry 209 is configured to provide a detection signal indicative of presence of the one or more bioactive agents in the surrounding medium based on determining the state change of the at least part of the receptor protein complexes 115 within the reaction cell 101.

The processing circuitry 209 and/or the sensing device 200 may include a processor, a memory, a data storage, a communication circuitry, a Bluetooth receiver and/or transmitter or other electronic components.

The sensing device 200 further comprises a heating element 210 for heating at least a part of the sensor chip 100. The heating element 210 can be located below the sensor chip 100, for example below the one or more sensors 207, or at some other position, where the temperature of the reaction cell can be controlled. The sensing device 200 may also include one or more temperature sensors for temperature control.

A channel 211 is formed in a housing of the sensing device 200 and passes along the surface of the sensor chip 100 where the open grid 113 is located. This channel 211 is connected to the surroundings by ventilation holes 212 or openings 212. Airflow through this channel 211 can optionally be driven by a ventilation device 213, such as a micro-ventilator 213.

Furthermore, the sensing device 200 provides one or more of an on/off button 214, a speaker 215, a connection for battery charging 216, one or more batteries, means for fixing the device on, for instance, backpack-straps 217, an array of diodes 218, that indicates the status of the device 200 and/or the sensor chip 100. Notifications on the device 200 and/or sensor chip 100 status include, but are not limited to battery status of the device 200, the conductivity of the aqueous solution or substrate 116 present in the reaction chamber of the sensor chip 100, usage of the device 200 or sensor chip 100, the remaining lifetime of the sensor chip 100, a predicted depletion of receptor protein complexes 115, or others.

Furthermore, the sensing device 200 comprises a user interface 219, for example a small screen 219, which gives information on the immediate and current air quality. Other information may be displayed in addition.

The working principle and operation of the sensing system 500, the sensing device 200 and/or the sensor chip 100 of FIGS. 1A to 2C are summarized in the following.

The sensor chip 100 can be removed from its packaging (not shown) and the blocking element 120 or pin 120 and the sealing cover (not shown) can be removed to activate the sensor chip 100. Once the pin 120 is removed, the channel 109 reaching through the aperture 108 is opened and water from the reservoir 102 will reach the semi-permeable membrane 121 and ultimately the reaction cell 101 containing a preferably lyophilized substrate 116 comprising salts, a buffer and optionally proteins that stabilize the receptor protein complexes 115 and, optionally, low amounts of a surface-active molecule. The osmolarity of the formed solution will draw water from the reservoir 102 or blister 118 into the reaction cell until the reaction cell 101 is completely filled and osmotic pressure is counter-balanced by the rigid housing of the reaction cell 101 or sensor chip 100.

The sensor chip 100 can then be inserted into the sensing device 200, fixed with the click-mechanism 204 present at the device 200 and the device 200 can be activated. When fixed within the sensing device 200, the contact between the electrodes 122 present within the chip 100 and the according connections located within the sensing device 200 are established and the optical connector 110 or aperture 110 present in the wall of the chip 100 is positioned in front of the light source 205 of the sensing device 200. Optionally, the heating element 210 can be activated. Once the sensor chip 100 has reached its operative status, for example a targeted osmolarity within the reaction cell 101 and a targeted minimal temperature, the ventilation device 213 and the light source 205 can be activated either automatically or by the user, for instance via an application installed on a user device, which may be wirelessly coupled to the sensing device 200.

The ventilation device 213 collects surrounding air and pushes it though the channel 211 in which the open grid 113 and the gas permeable membrane 114 of the sensor chip 100 are located. Molecules and bioactive agents present in the surrounding air will diffuse across the membrane 114 and trigger sensor responses as described hereinabove and with reference to FIGS. 3A to 3C.

FIGS. 3A to 3C each show a cross-sectional view of a reaction cell 101 of a sensor chip 100 for illustrating a possible working principle of the sensor chip 100.

The receptor protein complexes 115 are immobilized on a functional surface 182 of the reaction cell, such as on an inner surface of the gas-permeable membrane 114 by a low affinity ligand 301 that is covalently linked to the gas-permeable membrane 114. The receptor protein complexes 115 comprise at least one ligand binding domain 302 of a receptor protein configured to bind to one or more bioactive agents entering the reaction cell 101. The receptor protein complexes 115 further comprise at least one fluorescent label 303, which is also referred to as the fluorophore in the following, and an affinity tag 304.

The receptor protein complexes 115 or ligand binding domains 302 of receptor proteins may be full length receptor proteins or only one or several domains of receptor proteins, for example the ligand binding domain 302 only. The receptor protein complexes 115 or ligand binding domains 302 may include, for instance, a human xenosensor, hormone receptor protein, or an aryl hydrocarbon receptor.

The fluorophore 303 can, for instance, be the green fluorescent protein, in which case the receptor protein complexes 115 are a fusion protein, or a covalently linked fluorescent dye like, for instance, DAPI (4′,6-diamidino-2-phenylindole).

The affinity tag 304 has high affinity to a component 305 of a coating present on the light permeable wall 103 or detection window 103. The component 305 refers to or denotes the molecular trapping complexes 117 described with reference to FIGS. 1A to 1D. The affinity tag 304 may, for instance, be biotin, in which case the coating on the light permeable wall 103 or detection window 103 of the reaction cell 101 will comprise streptavidine. Alternatively, the affinity tag 304 may be a histidine tag, in which case the coating on the light permeable wall 103 of the reaction cell 101 will comprise chelated nickel ions.

The composition of the receptor protein complexes 115 can be custom tailored for a defined spectrum of detected bioactive environmental agents and a targeted sensitivity. For instance, more than one fluorophore 303 can be present in a single receptor protein complex 115, that is, different fluorophores and/or several units of the same fluorophore may be linked to the same receptor protein complex 115. In addition, more than one type of receptor protein complexes 115 can be present within a given sensor chip 100. For instance, different receptor proteins and/or different domains of the same receptor protein and/or receptor proteins of a different recombinant nature and/or receptor protein complexes of different composition, for example carrying different fluorophores, may be used.

Bioactive agents 307 or molecules 307 that are ligands of the receptor protein complexes 115 will diffuse through the gas permeable membrane 114 and, once in solution within the reaction cell 101, will compete with the low affinity ligand 301 covalently linked to the gas-permeable membrane 114. Depending on the relative affinities of the low affinity ligand 301 and the bioactive agent 307, this competitive binding will liberate the receptor protein complex 115 from the membrane 114 with a specific probability. The liberated receptor protein complex 308 in FIG. 3B diffuses freely in the reaction cell 101.

The light source 205 present in the sensing device 200 emits a beam or preferably a sheath of light 309 of wavelength λₑₓ into the reaction chamber, λₑₓ being the excitation wavelength of the fluorophores 303 present in in the receptor protein complexes 115. When the liberated receptor protein complex 308 diffuses into the light path, the fluorophore 303 is excited and emits fluorescent light of the wavelength λₑₘ as shown in FIG. 3B.

The light permeable wall 103 or detection window 103 of the sensor chip 100 and optionally a wall 208 separating the photodetectors 207 from the sensor chip 100 are permeable to light of wavelength λₑₘ. Due to the geometry of the reaction cell 101 and the location of the light sheath 309, approximately half of the light emitted by the fluorophores 303 reaches the sheath of photosensor 207 surrounding the reaction cell (the other half travels towards the non-permeable walls of the reaction cell or towards the gas permeable membrane and will not be detected).

Depending on the bandwidth of the photosensors 207, light of wavelength λₑₓ, which may be scattered by proteins or particulate impurities in the liquid matrix or substrate 116 within the reaction cell 101 may trigger a detection signal too. To avoid this, the detection window 103 of the reaction cell 101 is preferably impermeable to light of wavelength λₑₓ.

As the diffusional movement is random, receptor protein complexes 115 may stay in the light path or leave it and enter it again, giving several signals over time. After prolonged operation or high exposure of the sensor chip 100, a large fraction of the receptor proteins complexes 115 are liberated from the gas-permeable membrane 114, which may render a proper detection of additional fluorescence inaccurate. The light permeable wall 103 or detection window 103 of the reaction cell 101 therefore acts as a sink for receptor protein complexes 115, as it is coated with molecular trapping complexes 117, 305 that bind the affinity tag 304 on the receptor protein complexes 115 with high affinity.

Except for the light permeable wall 103 or detection window 103 of the reaction cell 101, all inner surfaces of the reaction cell can be covered with a coating of low protein adsorptivity and because of the inner geometry of the reaction cell 101, the large majority of the liberated receptor protein complexes 115 will eventually pass through the light path and reach the light permeable part 103 or detection window 103 of the cell 101 where they will be removed from the solution.

Operational parameters such as the mean residence time of a receptor protein complex 115 in the solution or its half-life can be calculated based on the mass and the size of the receptor protein complexes 115, the viscosity of the solution in the reaction cell and the temperature within the cell 101. This mean residence time may be used for correcting the detection signal reported by the sensing device 200 for multiple excitation and emission events and thereby increase the sensitivity and accuracy of the sensing device 200.

When scattered at, for instance, proteins or small particulate impurities present in the solution within the cell, light of wavelength λₑₓ may reach the immobilized receptor protein complexes 115 present at the gas-permeable membrane 114 or at the detection window 103 of the reaction cell, which are receptor protein complexes 115 that already have been liberated by bioactive agents 307 entering the reaction cell 101 and have diffused through the cell 101. This scattered light may excite the fluorophores 303 of the receptor protein complexes 115 and induce the emission of photons of wavelength λₑₘ, which may reach the photodetector 207 and hence generate signals not related to the immediate presence of air pollution. The coating on the light permeable wall 103 or detection window 103 of the reaction cell 101 and/or on the gas-permeable membrane 114 may therefore comprise molecules that are known to quench the fluorescence of the fluorophore 303 by electron transfer (not shown). Quenching of the scattered light will decrease and/or eliminate such off-site excitation events and thereby decrease and/or eliminate noise originating from light scattering. Alternatively, the fluorescent intensity detected by the photosensors 207 can be corrected for the scattering effects. For instance, the fluorescent intensity originating from receptor proteins trapped at the light permeable wall 103 or at the gas-permeable membrane 114 can be predicted based on the empirically determined amount of scattered light reaching them, the stability of the fluorophores 303 and the amount of receptor protein complexes 115 that are expected to be present there, which can be determined from the cumulative fluorescent signals detected by the photosensors 207, for example.

FIG. 4A shows a perspective view of a sensor chip 400 for detecting one or more bioactive agents. FIGS. 4B and 4C each show a cross-sectional view of the sensor chip 400 of FIG. 4A. Unless stated otherwise, the sensor chip 100 of FIGS. 4A and 4B comprises the same features as the sensor chip 100 described with reference to any of FIGS. 1A to 3C.

The sensory chip 400 of FIGS. 4A to 4C is a double-layered structure of cylindrical cross-section. Other cross-sections, such as a rectangular cross-section, are possible. The outer layer of the structure or sensor chip 400 comprises a rigid open grid 401 through which surrounding air may pass and enter an air chamber 402. The air chamber 402 is functionally analogous to the air channel 211 described above.

The inner layer of the structure of sensor chip 400 comprises an open grid 403 through which constituents present in the air within the air chamber 402 may access the gas permeable membrane 404. The inner open grid 403 and the gas permeable membrane 404 are functionally analogous to the open grid 113 and the gas permeable membrane 114 described above.

The volume confined within the cylinder formed by the gas permeable membrane 404 defines the reaction cell 405 of the sensor chip 400. It is functionally analogous to the reaction cell 101 described above.

In analogy to exemplary embodiment of the sensor chip 100 described above, the inner surface of the gas-permeable membrane 404 is covered with immobilized receptor protein complexes (not shown in FIGS. 4A to 4C).

Within the reaction cell 405, one or two or a multitude of optical guides 406 are located. These may be present in the form of fibers or in the form of a planar optical guide or any other suitable shape.

The optical guides 406 can be covered with covalently linked molecular trapping complexes that are known to bind one or several components the receptor protein complexes 115 with high affinity (not shown in FIG. 4). The distance between the gas permeable membrane 404 and the surface of the optical guides 406 is preferably in the range of 0.1 mm or below.

The chemical nature, the biological origin of the molecules or molecular complexes as well as the means of immobilization on the optical guides 406 are analogous to what is described for the molecules or molecular complexes 117 covalently linked to the light permeable wall 103 of the reaction cell 101 described with reference to the previous figures.

Alternatively, the arrangement of the immobilized receptor proteins and the high-affinity complexes may be inverted, that is, the receptor proteins may be immobilized on the optical guides 406 and the high affinity complexes may be immobilized on the gas permeable membrane 404.

Preferably, the absorption spectra and other optical properties of the immobilized molecules or molecular complexes as well as the absorption spectra of the covalent link do not or only to a limited extent overlap with the absorption spectra or other optical properties of the receptor protein complexes 115.

One end of the sensory chip 407, comprises a water reservoir 408, which is functionally analogous to the water reservoir 102 described above. The connection of the water reservoir 408 to the reaction cell 405 and the working principle of the water reservoir 408 may be identical to what is described above. A blocking element 409 or a removable pin 409 may seal a flexible blister 410 located within or defining the reservoir 408 from the lyophilized content (not shown) of the reaction cell 405. A semipermeable membrane 411 separates the substrate present within the reaction cell 405 from freely mixing with the water in the blister 410 once the pin 409 was removed.

Furthermore, said end of the sensor chip 400 may provide a reflexive element, for example an optical mirror, or an optical guide 412 that is able to reflect light passing through a single optical guide 406 and/or connects pairs of two optical guides 406.

The other end of the sensor chip 400 comprises a structure 413 or part 413 with one or two or a multitude of optical connectors 414 or apertures 414 which are aligned with and form extensions of the optical guides 406 located within the reaction cell 405 of the chip 400.

Furthermore, a portion of the structure 413 or part 413 of the sensor chip 400 comprises electrical contacts or connectors 415 that electrically connect the chip 400 to the sensing device when inserted therein. These contacts 415 are functionally analogous to the electrical contacts 122 described above.

When in operation, that is, when mounted in the activated sensing device, light 416 emitted by the optical system of the sensing device, for example by one or more light sources, passes longitudinally through the optical guides 406 spanning in the reaction cell 405 from one end of the chip 400 to the other end of the chip 400 along a longitudinal axis of the chip 400. The light is reflected or deflected by the reflexive element 412 or optical structure 412 and travels back through the optical guide 406 towards end or portion 413 of the sensor chip 400 and ultimately leaves the chip 400 towards the optical system of the sensing device, as shown by reference numeral 417 in FIG. 4B.

FIG. 5 shows a cross-sectional view of a sensing system 500 for detecting one or more bioactive agents. In particular, FIG. 5 show a sensing system 500 with a sensing device 550 and a sensor chip 400 inserted into the sensing device 550. Unless stated otherwise, the sensor chip 400 of FIG. 5 comprises the same features as the sensor chips 100, 400 described with reference to FIGS. 1A to 4C. Likewise, the sensing device 550 of FIG. 5 comprises the same features as the sensing device 200 described with reference to FIGS. 2A to 4C.

FIG. 5 shows the sensor chip 400 inserted into a sensory device 550. The structure 413 forming one end of the sensor chip 400 is plugged into the sensing device 550 and establishes contact with the electronic and optical connections located in the device 550. Specifically, the optical guides 406 present in the sensor chip 400 connect via the optical connectors 414 or apertures 414 to the optical system 503 located in the sensing device 550. The optical system 503 comprises one or several light sources 504 and one or several light detectors 505, such as photosensors 505.

The socket present at the device 550 and the end structure of the chip 400 provide complementary guides (not shown) that assure proper orientation of the chip 400 in the device 550, as described above. Fixation of the chip 400 in the socket can be achieved by, for instance, a click-in mechanism, a magnetic connection, or by screw-locking.

Furthermore, the device 550 comprises a battery, a microprocessor and potentially other electronic components, as described with reference to previous figures.

Surrounding air enters the air chamber 402 passively through the rigid open grid 401 of the sensor chip 400. Driving force may be movement of the surrounding air relative to the sensor chip 400 and/or Brownian motion.

Alternatively, the socket at the device 550 may be of the same length as the complete sensor chip 400 or of higher length than the sensor chip. In this case the chip 400 is completely inserted into the device 550 and the device 550 may comprise additional features, such as a ventilation device for sampling surrounding air and supplying it to the air chamber 402 of the sensor chip 500.

Features present on the surface of the device 550, such as diodes, a screen, or means for fixing the device on clothes, bikes or backpack straps are analogous to what is described with reference to FIGS. 2A to 2C.

FIG. 6 shows a cross-sectional view of a reaction cell 405 of a sensor chip 400 for illustrating a possible working principle of the sensor chip 400 of FIGS. 4A to 5.

Analogously to the example described with reference to FIGS. 1A to 3C, the receptor protein complexes 115 are immobilized at the gas-permeable membrane 404 by binding to covalently linked molecules 301 that are known to be low affinity ligands of the ligand binding domain 302 of the used receptor protein complexes 115.

Receptor protein complexes 115 consist of at least the ligand binding domain 302 of the receptor protein. Optionally, further components such as an affinity tag 304 are present. Features of the components of the receptor protein complexes 115, such as the biological origin or the recombinant nature, are as described above with reference to previous figures.

Environmental molecules or bioactive agents 307 present in the air chamber 402 diffuse through the open grid 403 and the gas-permeable membrane 404 and enter the reaction cell 405.

Once present in the reaction cell 405, the bioactive agents 307 compete for the binding at the ligand binding domain 302 of the receptor protein complexes 115 and eventually liberate the receptor protein complexes 115 from the membrane 404.

Liberated receptor protein complexes 308 are free to move within the reaction cell 405 and will eventually reach the optical guides 406 present in the center thereof and will bind to the molecules or molecular trapping complexes 117 present on the surface of the optical guides 406. This binding will change the properties of the boundary layer between the substrate or liquid matrix present in the reaction cell 405 and the surface of the optical guides 406.

Light 611 passing through the optical guide 406 is not entirely confined to the optical guide 406, but will penetrate into the substrate or liquid matrix present in the reaction cell 405 to a certain extent, forming an evanescent field which interacts with the immediate surroundings of the optical guide 406.

For example, the sensing device 550 or one or more sensors 505 can measure the absorption of the evanescent field. The light source present in the sensing device emits light of specified spectrum, for instance of a narrow window of wavelength λₑₘ. The absorption spectrum of one or several components of the receptor protein complexes 115 show a maximum at a wavelength of λ_{abs}. If λ_{abs}≈λₑₘ, the presence of receptor protein complexes 115 in close proximity to the surface of the optical guide 406 results in a decrease in the light intensity reaching the photosensor 505 in the sensor device 550. This decrease in light intensity can be quantified. The decrease per unit of time gives a direct measure for the number of receptor protein complexes 115 binding to the optical guide 406 per unit of time, which in turn is correlated to the amount of bioactive agents 307 entering the reaction cell 405 and hence to their concentration in the surrounding air. Alternative embodiments may for instance, employ waveguide coupling, surface plasmon resonance or fluorescent excitation of components of the receptor protein complexes 115 by the evanescent filed.

Alternatively, the detection principle may be inverse. Receptor protein complexes 115 may be immobilized on the optical guides 406 and upon binding bioactive agents that entered the reaction cell 405 from the surroundings, are liberated from the optical guides. Presence of ligands to the receptor protein complexes 115 will in this embodiment decrease the interaction between the complexes 115 and the evanescent field. For instance, the presence of ligands to the receptor protein complexes 115 in the surroundings will decrease the absorption of the evanescent light and hence increase the light intensity reaching the photodetectors 505 in the sensing device 550.

Yet another detection principle potentially employed in sensor chip 400 relies on piezoelectric detection of receptor protein complex deposition and/or liberation. This working principle can be identical to the one described with reference to FIG. 6, but detection of the binding of liberated receptor protein complexes 115 to high affinity ligands on the receiving surface is achieved by determining the mass of material bound to the surface instead of changes of the optical properties of the surface.

In such embodiments, the optical guides 406 can be replaced by one or more piezo elements able of detecting changes in the mass adsorbed to its surface with high sensitivity. The at least one piezo element may thereby be present in the center of the reaction cell 405 or comprise one or several of the walls of the reaction cell.

Alternatively, piezo-based sensor chips may rely on the inverse principle. The receptor protein complexes 115 can be immobilized on the piezo elements and are liberated therefrom when binding to bioactive agents that entered the reaction cell 405. The result will be the detection of a decrease in the mass bound to the piezo elements.

Whilst sensors for detecting environmental agents by piezo-elements have been described, the solution proposed here offers considerably higher sensitivity: Piezo-based sensors described in the art rely on measuring the mass increase brought by the binding of the environmental agent to, for instance, receptor proteins immobilized in the surface of the piezo element. The indirect detection method described herein, in contrast, relies on the binding of complete receptor protein complexes 115. These are commonly orders of magnitude higher in mass and can be detected more efficiently by the piezo elements.

In the following, exemplary materials for the sensor chip are described, which are not be construed limiting. The inner surface of the reaction cell, including the gas permeable membrane may be coated or manufactured from materials that are non-absorbent to (water soluble) proteins. Examples include fluoropolymers, such as Teflon, or highly hydrophobic coatings. Alternatively, a pre-coating of the surfaces with proteins such as ovalbumin or bovine serum albumin can be provided. The aperture through which light emitted by the light source enters the reaction cell can be permeable to light of at least wavelength λₑₓ.

Suitable materials for the gas permeable membrane may include, but are not limited to hydrophobic membranes with micron-sized perforations manufactured, for instance, from fluoropolymers, porous PET membranes, carbon paper, preferably with hydrophobic coating, and porous silicon-PDMS membranes.

The material of the detection window or light permeable wall of the reaction cell may be permeable to light of wavelength λₑₘ and preferably impermeable to light of wavelength λₑₓ. Corresponding materials are known from applications in photospectrometric instruments and will be specific to the fluorophores used in the reaction cell. The material of the detection window or light permeable wall of the reaction cell may further be resistant to aqueous salt solutions of neutral pH. If such materials with the additional spectroscopic properties are not available, a coated or double layered wall can be used. The coating or the protective layer may be permeable to least light of wavelength λₑₘ. To increase robustness of the detection window, a sandwich structure can be used: the material permeable to light of wavelength λₑₘ but impermeable to wavelength λₑₓ can, for example, be located between two layers of a robust material permeable to at least light of wavelength λₑₘ. Alternatively, a robust material permeable to at least light of wavelength λₑₘ can be coated with a suitable coating, which is permeable to light of wavelength λₑₘ, but not to light of wavelength λₑₓ

The optical system of the sensing device may include a light source that preferably emits light of a narrow bandwidth of wavelength in the range of λₑₓ, or λ_{abs} as described above and the aperture in the wall of the reaction cell may be permeable to light of at least this range of wavelengths. Alternatively, the light source may emit light of a broad range of wavelengths, in which case the aperture in the wall of the reaction cell is preferably permeable to light of a narrow bandwith in the range of λₑₓ or λ_{abs} only.

For optical guides, the material should be of suitable refractive index relative to the substrate or liquid matrix inside the reaction cell in order to optimize the intensity of the evanescent field. Exact values will depend on the used wavelength, which in turn depends on the absorption spectrum of the receptor protein complexes.

For the sensing device, one or more layers covering and protecting the sensors, such as the photosensors may be used. Requirements and technical solutions are identical to what is described for the light permeable wall or detection window hereinabove.

In the following, exemplary and non-limiting dimensions of the sensing device and the sensor chip are summarized.

For a stationary device, sensing device may have a volume of up to approximately 1000 cm³. The sensor chip may be approximately 1-10 cm in length, 5-10 cm in width and 2-3 cm in thickness, and the reaction cell may have a diffusion distance of preferably below 0.5 mm.

For a portable sensing device, the sensing device may have a volume of up to approximately 200 cm³. The sensor chip may be approximately 1-5 cm in length, 0.5-1 cm in width and 0.5-1 cm in thickness, and the reaction cell may have a diffusion distance of preferably below 0.5 mm.

FIG. 7 shows a flowchart illustrating a method of detecting one or more bioactive agents in a surrounding medium, for example using a sensing system, a sensing device and/or a sensor chip as described with reference to previous figures.

In a step S1 one or more bioactive agents are passed through a membrane 114 of the sensor chip 100 into a reaction cell 101 of the sensor chip 100.

In step S2, one or more bioactive agents are bound to one or more receptor protein complexes 115, thereby inducing a state change of at least a part of the receptor protein complexes 115.

In step S3, the induced state change of said at least a part of the receptor protein complexes 115 is detected.

In step S4, a detection signal indicative of presence of the one or more bioactive agents in the surrounding medium is generated based on the determined state change of said at least a part of the receptor protein complexes 115.

For the purpose of the present description and of the appended claims, except where otherwise indicated, all numbers expressing amounts, quantities, percentages, and so forth, are to be understood as being modified in all instances by the term “about”. Also, all ranges include the maximum and minimum points disclosed and include any intermediate ranges therein, which may or may not be specifically enumerated herein. In this context, therefore, a number A is understood as A±20% of A. Within this context, a number A may be considered to include numerical values that are within general standard error for the measurement of the property that the number A modifies. The number A, in some instances as used in the appended claims, may deviate by the percentages enumerated above provided that the amount by which A deviates does not materially affect the basic and novel characteristic(s) of the claimed invention. Also, all ranges include the maximum and minimum points disclosed and include any intermediate ranges therein, which may or may not be specifically enumerated herein.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art and practicing the claimed invention, from a study of the drawings, the disclosure, and the appended claims.

In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

1.-15. (canceled)

16. A sensor chip operatively couplable to a sensing device for detecting one or more bioactive agents in a surrounding medium, wherein the surrounding medium includes environmental air, the sensor chip comprising:
a reaction cell containing a plurality of receptor protein complexes; and
a membrane separating the reaction cell from the surrounding medium and being permeable for the one or more bioactive agents,
wherein the receptor protein complexes are configured to bind to the one or more bioactive agents in the reaction cell, such that a detectable state change of at least a part of the receptor protein complexes is induced, and
wherein the sensor chip is configured in shape and size to be at least partly removably inserted into a socket of the sensing device for detecting the one or more bioactive agents.

17. The sensor chip according to claim 16, wherein the detectable state change of said at least part of the receptor protein complexes is indicative of a presence of the one or more bioactive agents in the surrounding medium.

18. The sensor chip according to claim 16, wherein the state change of the at least part of receptor protein complexes is associated with one or more selected from the group consisting of a change in a conformational state of the at least part of receptor protein complexes, a change in a localization of the at least part of receptor protein complexes within the reaction cell, a change in a position of the at least part of receptor protein complexes within the reaction cell, a change in a composition of the at least part of the receptor protein complexes, a change in a mass of the at least a part of the receptor protein complexes, a change in a mass of at least a part of the reaction cell, a change in a physical property of the at least a part of the receptor protein complexes, a change in a physical property of at least a part of the reaction cell, a change in an optical property of at least a part of the reaction cell, a change in a chemical property of the at least a part of the receptor protein complexes, a change in a chemical property of a substrate contained in the reaction cell, a change in a conductivity of a substrate contained in the reaction cell, and a change in a concentration of free fluorescent or light absorbing molecules within at least a part of the reaction cell.

19. The sensor chip according to claim 16, wherein the receptor protein complexes are configured to change a position within the reaction cell upon binding to the one or more bioactive agents.

20. The sensor chip according to claim 16, wherein the surrounding medium further includes water.

21. The sensor chip according to claim 16,
wherein the membrane is permeable for gases, and/or
wherein the membrane is impermeable for water or aqueous liquid.

22. The sensor chip according to claim 16, further comprising one or more connectors configured to operatively couple the sensor chip to the sensing device.

23. The sensor chip according to claim 16, further comprising one or more electrodes at least partly arranged within the reaction cell and configured to determine a conductivity of a substrate or composition within the reaction cell.

24. The sensor chip according to claim 16, further comprising at least one detection window translucent for electromagnetic radiation emitted and/or scattered by at least one or more components of the receptor protein complexes.

25. The sensor chip according to claim 16,
further comprising at least one reservoir fluidly couplable with the reaction cell,
wherein the at least one reservoir is configured to supply de-ionized water to the reaction cell.

26. The sensor chip according to claim 16, wherein the receptor protein complexes each comprise at least one ligand binding domain of a receptor protein configured to bind to the one or more bioactive agents and configured to change conformation upon binding to one of the bioactive agents.

27. The sensor chip according to claim 26, wherein the receptor protein is a xenosensor protein or a hormone receptor protein.

28. A sensing device operatively couplable to at least one sensor chip according to claim 1 for detecting one or more bioactive agents in a surrounding medium, wherein the surrounding medium includes environmental air, the sensing device comprising:
at least one socket configured to at least partly receive the at least one sensor chip;
at least one sensor configured to determine a state change of at least a part of the receptor protein complexes in a reaction cell of the at least one sensor chip, the state change being induced by binding of one or more receptor protein complexes with one or more bioactive agents entering from the surrounding medium through a membrane of the sensor chip into the reaction cell of the sensor chip; and
processing circuitry coupled with the at least one sensor, wherein the processing circuitry is configured to provide a detection signal indicative of presence of the one or more bioactive agents in the surrounding medium based on determining the state change of the at least part of the receptor protein complexes.

29. The sensing device according to claim 28, wherein the detection signal is indicative of one or more selected from the group consisting of an amount of one or more bioactive agents per volume of surrounding medium, a mass of bioactive agent per volume of surrounding medium, a concentration of bioactive agent in the surrounding medium, a receptor protein activation potential of the surrounding medium, and a bioactivity of the surrounding medium.

30. A sensing system for detecting one or more bioactive agents in a surrounding medium, the system comprising:
at least one sensor chip according to claim 16; and
a sensing device operatively couplable to the at least one sensor chip and being configured to detect one or more bioactive agents in a surrounding medium, wherein the surrounding medium includes environmental air, the sensing device comprising:
at least one socket configured to at least partly receive the at least one sensor chip,
at least one sensor configured to determine a state change of at least a part of the receptor protein complexes in a reaction cell of the at least one sensor chip, the state change being induced by binding of one or more receptor protein complexes with one or more bioactive agents entering from the surrounding medium through a membrane of the sensor chip into the reaction cell of the sensor chip, and
processing circuitry coupled with the at least one sensor, wherein the processing circuitry is configured to provide a detection signal indicative of presence of the one or more bioactive agents in the surrounding medium based on determining the state change of the at least part of the receptor protein complexes.
